SEQUENCE LISTING (14 sequences)

SEQ ID NO: 1 - DNA, 572 bp, Human
ttggtcccaattagttggtggcggccaagccagcggcaggttccccccacccccggctcctcattaccgctggcggctcctaatgagcctggggagggggtgaccccggcgtccccggccccccggcctgcgtcactgcccggtgcgggggctgcggaggcgatataagggggctgccaccatcgctgccccagcccactgcacggtgatggagacccgccaggtgtccaggagccctcgggttcggctgctgctgctgctgctgctgctgctggtggtgccctggggcgtccgcactgcctcgggagtcgccctgcccccggtcggggtcctcagcctccgccccccaggacgggcctgggcggatcccgccacccccaggccgcggaggagcctggcgctggcggacgacgcggccttccgggagcgcgcgcggttgctggccgccctcgagcgccgccactggctgaactcgtacatgcacaagctgctggtgttggatgcgccctgagcgcgctgcccgtccccatcttaataaagaccatgccctgcgcaaaaaaaaaaaaaaaaaa

SEQ ID NO: 2 - protein, 100 aa, Human
METRQVSRSPRVRLLLLLLLLLVVPWGVRTASGVALPPVGVLSLRPPGRAWADPATPRPRRSLALADDAAFRERARLLAALERRHWLNSYMHKLLVLDAP

SEQ ID NO: 3 - protein, 100 aa, Mouse
METCQMSRSPRERLLLLLLLLLLVPWGTGPASGVALPLAGVFSLRAPGRAWAGLGSPLSRRSLALADDAAFRERARLLAALERRRWLDSYMQKLLLLDAP

SEQ ID NO: 4 - DNA, 20 bp, Primer
tgcgatcgatcgcgcttccg

SEQ ID NO: 5 - DNA, 26 bp, Primer
tcaagacaagaagaagctttgtgcat

SEQ ID NO: 6 - DNA, 24 bp, Primer
tgcatgtacgagttcagccagtgg

SEQ ID NO: 7 - DNA, 22 bp, Primer
cttccgggagcgcgcgcggttg

SEQ ID NO: 8 - DNA, 21 bp, Primer
ctagctgacgacgcggccttc

SEQ ID NO: 9 - DNA, 22 bp, Primer
gggcgcgtccagtagcaacagc

SEQ ID NO: 10 - DNA, 22 bp, Primer
cttgggtagccccctgtctcgg

SEQ ID NO: 11 - protein, 39 aa, Rat
SLALADDAAFRERARLLAALERRRWLDSYMQKLLLLDAP

SEQ ID NO: 12 - protein, 39 aa, Bovine
SLALADDAAFRERARLLAALERRHWLNSYMHKLLVLDAP

SEQ ID NO: 13 - protein, 39 aa, Mouse; identical to SEQ ID NO: 11

SEQ ID NO: 14 - protein, 39 aa, Human; identical to SEQ ID NO: 12

(19) United States
(12) Patent Application Publication
(10) Pub. No.: US 2004/0106773 A1
(43) Pub. Date: Jun. 3, 2004

(54) Human tuberoinfundibular peptide of 39 residues

(76) Inventors: Hao Wang, Rahway, NJ (US); Kenneth S Koblan, Rahway, NJ (US); Hong Sun, Rahway, NJ (US); Kimberly Della Penna, Rahway, NJ (US)

Correspondence Address: MERCK AND CO INC, P O BOX 2000, RAHWAY, NJ, 070650907

(21) Appl. No.: 10/399,602

(22) PCT Filed: Oct. 12, 2001
(86) PCT No.: PCT/US01/31954

(51) Int. Cl. C07K 14/47; C07H 21/04

(52) U.S. Cl. 530/324; 536/23.5; 435/69.1; 435/320.1; 435/325

(57) ABSTRACT

Disclosed herein are newly identified polynucleotides, polypeptides encoded by such polynucleotides, the use of such polynucleotides and polypeptides, as well as the production of such polynucleotides and polypeptides. More particularly, the polypeptide is a human protein, which has been putatively identified as a ligand of a parathyroid hormone receptor homolog, hereinafter referred to as PTH2 Receptor. In particular, the invention relates to isolated nucleic acid molecules, such as DNA and RNA encoding a novel human tuberoinfundibular peptide of 39 residues.

### BACKGROUND OF THE INVENTION

This invention relates to newly identified polynucleotides, polypeptides encoded by such polynucleotides, the use of such polynucleotides and polypeptides, as well as the production of such polynucleotides and polypeptides. More particularly, the polypeptide of the present invention is a human protein, which has been putatively identified as a ligand of a parathyroid hormone receptor homolog, hereinafter referred to as “PTH₂ Receptor”. In particular, the invention relates to isolated nucleic acid molecules, such as DNA and RNA encoding a novel human tuberoinfundibular peptide of 39 residues.

It is well established that many medically significant biological processes are mediated by proteins participating in signal transduction pathways that involve G-proteins and/or second messengers, e.g., cAMP (Lefkowitz, Nature 351:353-354 (1991)). Some examples of these proteins include the G-protein coupled receptor. (GPCR), such as those for adrenergic agents and dopamine (Kobilka, B. K., et al., PNAS 84:46-50 (1987); Kobilka, B. K., et al., Science 238:650-656 (1987); Bunzow, J. R., et al., Nature 336:783-787 (1988)), G-proteins themselves, effector proteins, e.g., phospholipase C, adenyl cyclase, and phosphodiesterase, and actuator proteins, e.g., protein kinase A and protein kinase C (Simon, M. I., et al., Science 252:802-8 (1991)).

The G protein transmembrane signaling pathways consist of three proteins: receptors, G proteins and effectors. G-protein coupled receptors are a diverse class of receptors that mediate signal transduction by binding to G proteins. These receptors are glycoproteins and comprise a superfamily of structurally related molecules. Possible relationships among seven transmembrane receptors are reviewed in Probst, et al., DNA and Cell Biology 11(1):1-20 (1992).

G-protein coupled receptors are known to share certain structural similarities and homologies (see, e-g., Gilman, A. G., Ann. Rev. Biochem. 56: 615-649 (1987), Strader, C. D., et al., The FASEB Journal 3:1825-1832 (1989), Kobilka, B. K., et al., Nature 329:75-79 (1985) and Young, et al., Cell 45:711-719 (1986)).

The G-protein coupled receptors exhibit detectable amino acid sequence similarity and all appear to share a number of structural characteristics including: an extracellular amino terminus; seven predominantly hydrophobic α-helical domains (of about 20-30 amino acids) connecting at least eight divergent hydrophilic loops and which are believed to span the cell membrane and are referred to as transmembrane domains 1-7; approximately twenty well-conserved amino acids; and a cytoplasmic carboxy terminus. The amino acid similarity among different G-protein receptors ranges from about 10% to more than 80% and receptors which recognize similar or identical ligands generally exhibit high levels of homology. The third cytosolic loop between transmembrane domains five and six is the intracellular domain responsible for the interaction with G-proteins. G-protein coupled receptors are found in numerous sites within a mammalian host.

The G-protein coupled receptors can be grouped based on their homology levels and/or the ligands they recognize. The G-protein coupled receptors includes dopamine receptors which bind to neuroleptic drugs used for treating psychotic and neurological disorders. Other examples of members of this family include calcitonin, adrenergic, endothelin, cAMP, adenosine, muscarinic, acetylcholine, serotonin, histamine, thrombin, kinin, follicle stimulating hormone, opsins, endothelial differentiation gene-1 receptor and rhodopsins, odorant, cytomegalovirus receptors, etc.

G-protein coupled receptors recognize a great diversity of ligands, e.g., neurotransmitters, peptide hormones and small molecules and transduce their signals via heterotrimeric guanine nucleotide-binding proteins (G-proteins), thereby effecting a broad array of biological activities through various intracellular enzymes, ion channels and transporters.

The function of GPCR activation is to stimulate GTP/GDP exchange at G proteins. In a cell, the guanine nucleotide exchange cycle is initiated by binding of an agonist—occupied (or activated) GPCR to a heterotrimeric C-protein in the cell membrane. This stimulates the dissociation of the GDP from the α-subunit of the G-protein, thereby allowing endogenous GTP to bind in its place. This, in turn, causes dissociation of the receptor and the Gα-GTP and Gβr-subunits of the G-protein. The Gα-GTP and Gβr-subunits can each activate effectors, such as adenyl cyclase, phospholipase C, and ion channels. The Gα-GTP is inactivated by intrinsic GTPase, which hydrolyzes the GTP to GDP; Gα-GDP in turn inactivates the Gβr by binding to it, thereby resulting in an inactive GDP-containing heterotrimeric G-protein ready for the next activation cycle.

Thus, the function of each G-protein coupled receptor is to discriminate its specific ligand from the complex extracellular milieu and then to activate G-proteins to produce a specific intracellular signal. In summary, cell surface proteins, by intracellularly transmitting information regarding the extracellular environment via specific intracellular pathways induce an appropriate response to a particular stimulus. Indeed, by virtue of an array of varied membrane surface proteins, eukaryotic cells are exquisitely sensitive to their environment.

The parathyroid hormone/parathyroid hormone-related protein (PTH/PTHrP) receptor is a member of a subgroup of the G protein-coupled receptor superfamily that includes the receptors for glucagon, glucagon-like peptide-1, vasoactive intestinal protein, CRF, secretin, calcitonin (CT), and a number of others. The two ligands for the PTH/PTHrP receptor, PTH and PTHrP, are the products of distinct, yet evolutionarily related, genes (Behar, V., et al., Endocrinology 137:2748-2757 (1996)).

PTH is secreted by four small glands located behind the thyroid gland. The most important physiological function of PTH is to maintain extracellular fluid calcium concentration by increasing the rate of bone destruction with mobilization of calcium and phosphate from bone, increasing renal tubular resorption of calcium, increasing intestinal absorption of calcium and decreasing renal tubular resorption of phosphate. These actions account for all-important clinical manifestations of PTH excess or deficiency (Behar, supra).

Regulation of calcium concentration is necessary for the normal function of the gastrointestinal, skeletal, neurologic, neuromuscular, and cardiovascular systems. PTH regulates calcium and phosphate metabolism via the activation of G-protein coupled receptor that also binds PTHrP. This dual hormone recognition is presumed to be via a unique seven-transmembrane domain receptor (PTH/PTHrP receptor) that specifically recognizes the N-terminal regions 1-34 of both hormones (Behar, supra).

PTH synthesis and release are controlled principally by the serum calcium level: a low level stimulates and a high level suppresses both the hormone synthesis and release. PTH exerts its effects primarily through receptor-mediated activation of adenylate cyclase, although receptor-mediated activation of phospholipase C by PTH has also been reported (Hruska, et al., J. Clin. Invest. 79:230 (1987)).

Recently, a new G-protein coupled receptor (PTH₂ receptor) was identified during a molecular screen for members of the secretin receptor family. A number of previous studies have suggested that PTH receptors with properties different from those of the PTH/PTHrP receptor exist. The human PTH₂ receptor shares 51% identity (over 70% sequence similarity) with the human PTH₁ receptor (also referred to as the PTH/PTHrP receptor) as well as significant homology with the other receptors of this class (Usdin, T. B., Endocrinology 138: 831-834 (1997)).

The PTH₂ receptor is a G protein-coupled receptor selectively activated by PTH (1). It is a member of the secretin receptor family, which includes receptors for secretin, vasoactive intestinal polypeptide, pituitary adenylate cyclase activating polypeptide, calcitonin, glucagon, glucagon-like peptide I, gastric inhibitory polypeptide, and CRF as well as PTH and PTHrP (originally called hypercalcemia of malignancy factor) (Usdin, T. B., et al., Endocrinology 137:4285-4297 (1996)).

Both PTH receptors, PTH₁ and PTH₂ receptors belong to the type II family of G-protein-coupled receptors that respond to peptide modulators, including calcitonin, glucagon, secretin, and vasoactive intestinal polypeptide. The similarity identified for PTH receptors extends to their ligands. (Usdin, T. B., et al., Endocrinology 140:3363-3371 (1999)).

The PTH₂ and PTH₁ receptors, together with their ligands, have presumably evolved to selectively mediate different physiological functions. In this regard, the PTH₁ receptor mediates the principal actions of PTH (elevation of blood calcium levels) and PTHrP (a locally acting autocrine/paracrine factor and developmental regulator) whereas the PTH₂ receptor responds to TIP39 and perhaps PTH but not to PTHrP. (Hoare, S. R., J. Bio. Chem. 275: 27274-27283 (2000)).

Many of the cells that express the PTH₂ receptor are hormone secreting, and the colocalization with somatostatin is striking. It is suggested that the PTH₂ receptor may be involved in the specified regulatory functions of each of the cells that express it. (Usdin, T. B., et al., Endocrinology 140:3363-3371 (1999)).

The newly discovered PTH₂ receptor is most abundant in the nervous system, particularly, in the brain. Its expression is relatively high in the hypothalamus, where nerve terninals in the median eminence and cell bodies in the periventricular nucleus have particularly high receptor levels, suggesting a role in the modulation of pituitary function. It is present at low levels in the placenta and testis. In many of the areas where PTH₂ receptor mRNA is present, it is clear from the size and morphology of the labeled cells that it is present within neurons. This and the fact that it is present in distinct brain nuclei suggest that it may function as a neurotransmitter receptor (Ted. B. Usdin, Endocrinology 138: 831-834 (1997)).

PTH₂ receptor concentration in the superficial lamina of the spinal cord dorsal horn suggests a role in the modulation of pain perception. In the periphery, the receptor is expressed by discrete cells in a number of tissues including pancreatic islet somatostatin cells, heart and vascular muscle cells, and cells within bronchioles and vasculature in the lung (Usdin, T. B., et al., Nature Neuroscience 2:941-943 (1999); Hoare, supra).

There are some reports that suggest that the PTH₂ receptor is present in some of the phylogenetically older parts of the forebrain as well as in several hypothalamic and brainstem nuclei, suggesting that it is involved in the modulation of primitive functions. Several of the areas where PTH₂ receptor mRNA is most abundant contain a major or primarily cholinergic population of neurons, raising the possibility that activation of this receptor modulates cholinergic neurotransmission. Likewise, it is striking that many of the areas that express PTH₂ receptor mRNA are part of hippocampal input or output pathways (Usdin, T. B., et al., Endocrinology 137:4285-4297 (1996)).

PTH₂ encoding messenger RNA is also abundantly expressed in arterial and cardiac endothelium and at lower levels in vascular smooth muscle. It is also abundant in the lung, both within bronchi and in the parenchyma, and is present within the exocrine pancreas. It is expressed by sperm in the head of the epididymis. A small number of cells associated with the vascular pole of renal glomeruli also express. the receptor. These data suggest that this receptor may be responsible for PTH effects in a number of physiological systems.

The location of PTH₂ receptor mRNA in the cardiovascular system and kidney suggests that it may play a role in blood pressure regulation. Indeed, elevated PTH, especially in chronic renal failure, has been reported to cause deleterious effects in the heart and lung. Stage-specific expression in sperm raises the question of whether it is involved in male infertility. The data suggest that increased PTH may play a role in several chronic diseases and because of the possibility that the PTH₂ receptor is involved. (Usdin, T. B., et al., Endocrinology 137:4285-4297 (1996)).

To date, PTH was thought to be the only endogenous substance known to activate the PTH₂ receptor, so it was assumed to be the natural ligand. Recently a putative endogenous ligand for PTH₂ was isolated and characterized. The 39-amino acid peptide, named tuberoinfundibular peptide or TIP39, has been isolated from bovine hypothalamus, and based on the PTH₂ receptor distribution it is thought to be involved in modulation of pain and pituitary function. The PTH receptor and bovine TIP39 are considered to form a part of an extended family of related receptors and ligands (1). Usdin, T. B., et al., Nature Neuroscience, 2:941-943 (1999).

TIP39 appears to be distantly related to PTH and PTHrP. Clues about the biological function of the PTH₂ receptor and TIP39 are provided by the cellular distribution of the receptor. Strong staining using an antibody that recognizes the PTH₂ receptor has been observed in the external zone of the median eminence, where hypothalamic neurons release factors into the portal circulation that regulate pituitary hormone secretion (T. B. Usdin, TiPS 21: 128-130 (2000)).

Bovine TIP39 has been implicated as a good candidate as the PTH₂ receptor's endogenous ligand. Indeed, it has been shown to be a strong activator of the human, rat, and zebrafish PTH₂ receptors. According to investigators, because TIP39, used in various studies, was purified from bovine brain and PTH₂ receptor expression is highest in the brain, it appears likely that, at least in the CNS, a homolog of bovine TIP39 acts on the human PTH₂ receptor (Usdin, T. B., Endocrinology 140:3363-3371 (1999); Hoare, supra).

Studies by T. B. Usdin have shown that bovine tuberoinfundibular peptide of 39 residues (bTIP39) activates the human and rat PTH₂ receptors (EC₅₀=0.5 and 0.8 nM, respectively) but has little or no effect at PTH₁ receptors. Activation of the rat PTH₂ receptor by bTIP39 results in a twofold greater accumulation in cAMP than that elicited by PTH, and bTIP39 is 100-fold more potent than PTH. By contrast, bTIP39 and PTH have a similar potency and efficacy at the human PTH₂ receptor. (T. B. Usdin, TiPS 2:128-130 (2000)).

As noted, the PTH₂ receptor has been observed to be present in several populations of hypothalamic neurons, and its expression in the somatostatin-containing cells of the periventricular nucleus, which are major regulators of growth hormone secretion, is particularly striking. The PTH₂ receptor has also been shown to be expressed in primary sensory neurons (Usdin, T. B., Endocrinology 140:3363-3371 (1999)).

According to conventional data, bovine TIP39 increases cAMP in dorsal root ganglion (DRG)-like F-11 cells, and because other agents that increase cAMP in DRG neurons potentiate nociception, it is suggested that a PTH₂ receptor antagonist might be useful for ameliorating some types of pain.

As well, it is hypothesized that the PTH₂ receptor might stimulate somatostatin release in both pancreatic islets and the hypothalamus, and thus therapeutic agents/compounds that are selective for the PTH₂ receptor might indirectly modulate secretion of insulin, glucagon or growth hormone (T. B. Usdin, TiPS 21: 128-130 (2000)).

Discrete cellular populations in several peripheral organs have relatively high PTH₂ receptor expression. These include somatostatin-synthesizing D cells of the pancreatic islets, calcitonin-synthesizing parafollicular C cells of the thyroid, several populations of gastrointestinal peptide-synthesizing cells, and cartilage and heart muscle cells. As such, the data suggest that the PTH₂ receptor and bovine TIP39 could be involved in the modulation of pituitary hormone release, sensory and particularly nociceptive sensitivity, pancreatic islet function, Ca²⁺ homeostasis and cardiovascular function (Usdin, T. B., et al., Nature Neuroscience 2:941-943 (1999)).

As such, there are many potential pharmacological uses for compounds that interact with and modulate the activity of cell surface proteins such as the PTH₂ receptor. For example, calcium channels, play a central role in regulating intracellular Ca²⁺ levels, which influence cell viability and function. Intracellular levels of Ca²⁺ concentrations are implicated in a number of vital processes in animals, such as neurotransmitter release, pain, muscle contraction, pacemaker activity, and secretion of hormones and other substances.

In order to study the function of human TIP39 and to obtain disease-specific pharmacologically active agents, there is a need to obtain isolated (preferably purified) human TIP39, and isolated (preferably purified) human TIP39. In addition, there is also a need to develop assays to identify such pharmacologically active agents.

As such, the availability of the disclosed isolated nucleic acid molecules that encode human TIP39 will fulfill the above referenced voids in the prior art and will provide detailed information of the human TIP39 structure and function based on predictions drawn from non-human TIP39 data. This, in turn, will allow for the development of therapeutic candidates that modulate pain perception, treat metabolic disorders, hypertension, cardiovascular disease and neurological disorders attending a defective human TIP39 or its respective receptor, etc.

As well, the identity of a human TIP39 would enable the rapid screening of a large number of compounds to identify those candidates suitable for further, in-depth studies of therapeutic applications.

### SUMMARY OF THE INVENTION

The invention provides isolated nucleic acid molecules encoding a novel human protein, including mRNAs, DNAs, cDNAs, genomic DNA as well as antisense analogs thereof and biologically active and diagnostically or therapeutically useful fragments thereof.

Another aspect of the invention provides an isolated, purified human tuberoinfundibular peptide of 39 residues (invention polypeptide/peptide), which is a ligand for the human parathyroid hormone-2 receptor.

Plasmids containing DNA encoding the invention peptide are also provided. Recombinant cells containing the above-described DNA, MRNA or plasmids are also provided herein.

In accordance with a further aspect of the present invention, there are provided processes for producing the invention peptide(s) by recombinant techniques comprising culturing transformed prokaryotic and/or eukaryotic host cells, containing nucleic acid sequences encoding the invention peptide under conditions promoting expression of the invention peptide, followed by subsequent recovery of the polypeptide(s).

In accordance with yet another further aspect of the present invention, there are provided antibodies against the invention peptide.

In accordance with still another embodiment of the invention, there are provided processes of administering compounds comprising the invention peptide to a host that bind to and activate the human parathyroid hormone-2 receptor.

In accordance with yet another aspect of the present invention, there are provided nucleic acid probes comprising nucleic acid molecules of sufficient length to specifically hybridize to the polynucleotide sequences of the present invention.

In another aspect, the invention features assays for detecting the invention peptide.

In accordance with still another aspect of the present invention, there are provided diagnostic assays for detecting diseases related to mutations in the nucleic acid sequences encoding the invention peptide and for detecting an altered level of the encoded polypeptide.

In accordance with yet a further aspect of the present invention, there are provided processes for utilizing the invention peptide or nucleic acid molecules encoding such polypeptides for in vitro purposes such as synthesis of DNA and manufacture of DNA vectors.

A further aspect of the invention provides assay(s) for screening and identifying potential pharmaceutically effective compounds that specifically interact with and modulate the activity of cell surface proteins, particularly PTH₂ receptor.

In a related aspect, the invention features fragments of the invention peptide. Preferably, the fragment is capable of binding human PTH₂ receptor. In preferred embodiments, this fragment is at least six amino acids long or its analog, which is capable of binding PTH₂ receptor, wherein “analog” denotes a peptide having a sequence at least 50% (and preferably at least 70%) identical to the peptide of which it is an analog.

Also within the invention is a therapeutic composition including, in a pharmaceutically-acceptable carrier, (a) the invention peptide, (b) an immunologically active or biologically active fragment thereof, or (c) an antibody having affinity for (a) or (b) above. These therapeutic compositions provide a means for treating various disorders characterized by abnormal (low or ubiquitous) level of the invention peptide or a dysfunctional PTH₂ receptor.

These invention nucleic acids, invention peptides and antibodies, including fragments thereof are useful as diagnostics, for distinguishing disease states caused by a dysfunctional endogenous human TIP39 or PTH₂ receptor from those which are not.

The nucleic acid probes of the invention enable one of ordinary skill in the art of genetic engineering to identify and clone similar polypeptides from any species thereby expanding the usefulness of the sequences of the invention. As well, the sequences of the invention will enable one skilled in the art to screen for and identify other ligands of the PTH₂ receptor in human sand other mammalian species.

Also provided in accordance with the present invention are methods for identifying cells that express the invention peptide. Methods for identifying compounds which modulate the activity of the invention peptide are also provided.

The DNA, mRNA, vectors, and cells provided herein permit production of human tuberoinfundibular peptide of 39 residues, as well as antibodies to the peptide. This provides a means to prepare synthetic or recombinant invention peptides that are substantially free of contamination from many other proteins whose presence can interfere with analysis of a single human tuberoinfundibular peptide of 39 residues. The availability of desired receptors, i.e., PTH₂R makes it possible to observe the effect of a drug substance on the receptor and to thereby perform initial in vitro screening of the drug substance in a test system that is specific for the invention peptide and its corresponding receptor.

The availability of human TIP39-specific antibodies makes possible the application of the technique of immunohistochemistry to monitor the distribution and expression density of the invention peptide as well as its corresponding receptor (e.g., in normal vs. diseased brain tissue). Such antibodies could also be employed for diagnostic and therapeutic applications. This antibody is preferably capable of neutralizing a biological activity of the PTH₂ receptor (i.e. adenylate cyclase activation).

Thus, antibodies, (monoclonal or polyclonal), including purified preparations of an antibody, which is capable of forming an immune complex with the invention peptide, such antibody being generated by using as antigen either (1) a polypeptide that includes a fragment of invention peptide, or (2) the invention peptide.

The ability to screen drug substances in vitro to determine the effect of the drug on native human tuberoinfundibular peptide of 39 residues or its binding to its native receptor should permit the development and screening of TIP39-specific or disease-specific drugs. Also, testing of the invention peptide with a variety of potential agonists or antagonists provides additional information with respect to the function and activity of the invention peptide and should lead to the identification and design of compounds that are capable of very specific interaction with native human tuberoinfundibular peptide of 39 residues or its interaction with its specific receptor. The resulting drugs should exhibit fewer unwanted side effects than drugs identified by screening with cells that express a non-human TIP39.

Further in relation to drug development and therapeutic treatment of various disease states, the availability of DNAs encoding the invention peptide enables identification of any alterations in such genes (e.g., mutations) which may correlate with the occurrence of certain disease states. In addition, the creation of animal models of such disease states becomes possible, by specifically introducing such mutations into synthetic DNA sequences which can then be introduced into laboratory animals or in vitro assay systems to determine the effects thereof.

Therefore, it is an object herein to isolate and characterize nucleic acid molecules encoding human TIP39. It is also an object herein to provide methods for the recombinant production of the invention peptide as well as to provide methods for screening compounds to identify compounds that modulate the activity of human TIP39.

Other features and advantages of the invention will be apparent to those of skill in the art upon further study of the specification and claims.

### BRIEF DESCRIPTION OF THE DRAWINGS

FIG. 1 presents the nucleotide and deduced amino acid sequence of human tuberoinfundibular peptide of 39 residues. The deduced amino acid sequence is that of the prepolypeptide, i.e., immature/precursor or prior to post-translational modification. The bold sequence around the ATG (GCACGGT atgG) partially conformed to Kozak's rule (GCACACCatgG). Polyadenylation signal AATAA is underlined. The deduced amino acid sequence refers to the polypeptide sequence prior to post-translational modification and hence it is labeled “precursors”.

FIG. 2 presents depicts the alignment of the polypeptide sequences of human tuberoinfundibular peptide of 39 residues precursors (prior to post translational modification) and its corresponding mouse equivalent. Importantly, the predicted signal peptide sequence in the human sequence (SEQ ID NO:2) is single-underlined, while the predicted mature sequence, is double underlined.

FIG. 3 depicts the alignment of the mature polypeptide amino acid sequences corresponding to human tuberoinfundibular peptide of 39 residues and its corresponding rat, mouse and bovine equivalent. Importantly, the amino acid sequence of human TIP39 shown in this figure corresponds to the mature protein, i.e., after post translational modification etc.

FIG. 4A and FIG. 4B depict dose-response curve analysis of the effect of human and mouse tuberoinfundibular peptide of 39 residues, human PTH and rat PTH on rat PTH₂R (A) and human PTH₂R (B) expressing HEK293 cells. The dose-response effect is measured by an increase in cAMP levels after stimulation with a prospective ligand.

FIG. 5A and FIG. 5B illustrate a dose-response curve of agonists on stably-transfected HEK293 cells expressing a rat PTH₂R (A) and human PTH₂R (B) respectively. A representative example of the dose-response effect of potential agonists is to increase intracellular calcium concentration.

### DETALED DESCRIPTION OF THE INVENTION

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described.

All publications mentioned herein are incorporated herein by reference for the purpose of describing and disclosing the methodologies, vectors etc which are reported in the publications that might be used in connection with the invention. Nothing herein is to be construed as an admission that the invention is not entitled to antedate such disclosure by virtue of prior invention.

In the description that follows, a number of terms used in the field of recombinant DNA technology are extensively utilized. In order to provide a clearer and consistent understanding of the specification and claims, including the scope to be given such terms, the following definitions are provided.

The present invention provides isolated nucleic acid molecules that encode a novel human peptide, human tuberoinfundibular peptide of 39 residues (hTIP39). Specifically, isolated DNA encoding the invention peptide are described as are recombinant messenger RNA (mRNA). Splice variants of the isolated DNA are also described. Typically, unless hTIP39 arises as a splice variant, hTIP39-encoding DNA will share substantial sequence homology (i.e., greater than about 90%), with the hTIP39-encoding DNA described herein. DNA or RNA encoding a splice variant may share less than 90% overall sequence homology with the DNA or RNA provided herein, but such a splice variant would include regions of nearly 100% homology to the disclosed DNAs.

“Invention nucleic acid(s)” and “nucleic acid molecules” are used interchangeably and refer to the nucleic acid molecules of the invention that encode the invention peptide.

As used herein, “humanTIP39” or “hTIP39” refers to a human TIP39 that is encoded by a nucleic acid molecule that hybridizes under high stringency conditions to the nucleotide sequences disclosed herein. Such nucleic acid molecule can be characterized in a number of ways, for example—the DNA may encode the amino acid sequence set forth in SEQ ID NO:2 or 14, or the DNA may include the nucleotide sequence as set forth in SEQ ID NO:1.

The nucleic acid molecules described herein are useful for producing invention peptides, when such nucleic acids are incorporated into a variety of protein expression systems known to those of skill in the art. In addition, such nucleic acid molecules or fragments thereof can be labeled with a readily detectable substituent and used as hybridization probes for assaying for the presence and/or amount of a hTIP39 encoding gene or mRNA transcript in a given sample. The nucleic acid molecules described herein, and fragments thereof, are also useful as primers and/or templates in a PCR reaction for amplifying genes encoding the invention protein described herein.

A “gene” refers to a nucleic acid molecule whose nucleotide sequence codes for a polypeptide molecule. Genes may be uninterrupted sequences of nucleotides or they may include such intervening segments as introns, promoter regions, splicing sites and repetitive sequences. A gene can be either RNA or DNA. A preferred gene is one that encodes the invention peptide.

The term “nucleic acid” or “nucleic acid molecule” is intended for ribonucleic acid (RNA) or deoxyribonucleic acid (DNA), probes, oligonucleotides, fragment or portions thereof, and primers. DNA can be either complementary DNA (cDNA) or genomic DNA, e.g. a gene encoding the invention peptide.

Unless otherwise indicated, a nucleotide defines a monomeric unit of DNA or RNA consisting of a sugar moiety (pentose), a phosphate group, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose, it is referred to as a nucleotide. A sequence of operatively linked nucleotides is typically referred to herein as a “base sequence” or “nucleotide sequence”, and their grammatical equivalents, and is represented herein by a formula whose left to right orientation is in the conventional direction of 5′-terminus to 3′-terminus.

Each “nucleotide sequence” set forth herein is presented as a sequence of deoxyribonucleotides (abbreviated A, G, C and T). However, by “nucleotide sequence” of a nucleic acid molecule is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U). For instance, reference to an RNA molecule having the sequence of SEQ ID NO:1 set forth using deoxyribonucleotide abbreviations is intended to indicate an RNA molecule having a sequence in which each deoxyribonucleotide A, G or C of SEQ ID NO:1 has been replaced by the corresponding ribonucleotide A, G or C, and each deoxyribonucleotide T has been replaced by a ribonucleotide U.

A “fragment” of a nucleic acid molecule or nucleotide sequence is a portion of the nucleic acid that is less than full-length and comprises at least a minimum length capable of hybridizing specifically with the nucleotide sequence of SEQ ID NO:1 under stringent hybridization conditions. The length of such a fragment is preferably 15-17 nucleotides or more.

A “variant” nucleic acid molecule or DNA molecule refers to DNA molecules containing minor changes in the native nucleotide sequence encoding the invention polypeptide(s), i.e., changes in which one or more nucleotides of a native sequence is deleted, added, and/or substituted, preferably while substantially maintaining the biological activity of the native nucleic acid molecule. Variant DNA molecules can be produced, for example, by standard DNA mutagenesis techniques or by chemically synthesizing the variant DNA molecule or a portion thereof. Generally, differences are limited so that the nucleotide sequences of the reference and the variant are closely similar overall and, in many regions, identical.

Changes in the nucleotide sequence of a variant polynucleotide may be silent. That is, they may not alter the amino acids encoded by the polynucleotide. Where alterations are limited to silent changes of this type, a variant will encode a polypeptide with the same amino acid sequence as the reference.

Alternatively, the changes may be “conservative”. Conservative variants are changes in the nucleotide sequence that may alter the amino acid sequence of a polypeptide encoded by the reference polynucleotide. Such nucleotide changes may result in amino acid substitutions, additions, deletions, fusions and truncations in the polypeptide encoded by the reference sequence. Thus, conservative variants are those changes in the protein-coding region of the gene that result in conservative change in one or more amino acid residues of the polypeptide encoded by the nucleic acid sequence, i.e. amino acid substitution.

An “insertion” or “addition”, as used herein, refers to a change in an amino acid or nucleotide sequence resulting in the addition of one or more amino acid or nucleotide residues, respectively, as compared to the naturally occurring molecule.

A “substitution”, as used herein, refers to the replacement of one or more amino acids or nucleotides by different amino acids or nucleotides, respectively.

Preferably, a variant form of the preferred nucleic acid molecule has at least 70%, more preferably at least 80%, and most preferably at least 90% nucleotide sequence similarity with the native gene encoding the invention peptide.

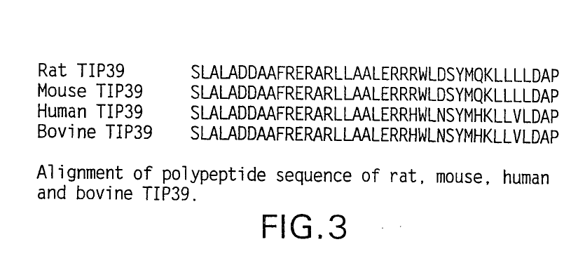

“Mature” protein as it relates to the human TIP39 disclosed herein and shown in FIG. 3, refers to the mature protein(s) after post-translational modification. In the same vein, “precursors” or “precursor” or “prepolypeptide” refers to the deduced amino acid sequence of the gene product of the nucleic acid molecule encoding a human TIP39 prior to any post-translational modification. “Primer” or “nucleic acid polymerase primer(s)” refers to an oligonucleotide, whether natural or synthetic, capable of acting as a point of initiation of DNA synthesis under conditions in which synthesis of a primer extension product complementary to a nucleic acid strand is initiated, i.e., in the presence of four different nucleotide triphosphates and an agent for polymerization (i.e., DNA polymerase or reverse transcriptase) in an appropriate buffer and at a suitable temperature. The exact length of a primer will depend on many factors, but typically ranges from 15 to 25 nucleotides. Short primer molecules generally require cooler temperatures to form sufficiently stable hybrid complexes with the template. A primer need not reflect the exact sequence of the template, but must be sufficiently complementary to hybridize with a template. A primer can be labeled, if desired.

“Polypeptide” or “peptide” or “protein” refers to a polymer of amino acid residues and to variants and synthetic analogs of the same and are used interchangeably herein. Thus, these terms apply to amino acid polymers in which one or more amino acid residues is a synthetic non-naturally occurring amino acid, such as a chemical analog of a corresponding naturally occurring amino acid, as well as to naturally occurring amino acid polymers. The invention peptide is the preferred polypeptide.

The term “amino acid sequence” as used herein refers to an oligopeptide, peptide, polypeptide, or protein sequence, and fragments or portions thereof, and to naturally occurring or synthetic molecules.

“Identity” or “homology” with respect to the invention peptide is defined herein as the percentage of amino acid residues in the candidate sequence that are identical with the residues in SEQ ID NO: 2 or 14, preferably SEQ ID NO:14, corresponding to a homosapien, after aligning the sequences and introducing gaps, if necessary, to achieve the maximum percent homology, and not considering any conservative substitutions as part of the sequence identity. No N- nor C-terminal extensions, deletions nor insertions shall be construed as reducing identity or homology.

As used herein, a “variant” of the invention peptide refers to a polypeptide having an amino acid sequence with one or more amino acid substitutions, insertions, and/or deletions compared to the sequence of the invention peptide. Generally, differences are limited so that the sequences of the reference (invention peptide) and the variant are closely similar overall, and in many regions, identical. Such variants are generally biologically active and necessarily have less than 100% sequence identity with the polypeptide of interest.

In a preferred embodiment, the biologically active variant has an amino acid sequence sharing at least about 70% amino acid sequence identity with the invention peptide—SEQ ID NO: 14., preferably at least about 75%, more preferably at least about 80%, still more preferably at least about 85%, even more preferably at least about 90%, and most preferably at least about 95%. Amino-acid substitutions are preferably substitutions of single amino-acid residues.

A “fragment” of the invention peptide (reference protein) is meant to refer to a protein molecule which contains a portion of the complete amino acid sequence of the wild type or reference protein.

Complementary DNA clones encoding the invention peptide may be prepared from the DNA provided. The nucleic acid clones provided herein may be used to isolate genomic clones encoding the invention peptide and to isolate any splice variants by screening libraries prepared from different neural tissues.

Alternatively, the library may be screened with a suitable probe. Thus, one means of isolating a nucleic acid encoding the invention peptide is to probe a mammalian genomic library with a natural or artificially designed nucleic acid probe using methods well known in the art. Nucleic acid probes derived from the invention peptide encoding gene(s) are particularly useful for this purpose. Examples of nucleic acids are RNA, cDNA, or isolated genomic DNA encoding the invention peptide. Such nucleic acids may include, but are not limited to, nucleic acids having substantially the same nucleotide sequence as set forth in SEQ ID NO:1 or one encoding the amino acid sequence as set forth in SEQ ID NO: 2 or 14, preferably SEQ ID NO: 14.

Nucleic acid amplification techniques, which are well known in the art, can be used to locate splice variants of the invention peptide. This is accomplished by employing oligonucleotides based on DNA sequences surrounding divergent sequence(s) as primers for amplifying human RNA or genomic DNA. Size and sequence determinations of the amplification products can reveal the existence of splice variants. Furthermore, isolation of human genomic DNA sequences by hybridization can yield DNA containing multiple exons, separated by introns that correspond to different splice variants of transcripts encoding the invention peptide. Techniques for nucleic-acid manipulation are described generally in, for example, Sambrook, et al. (1989) and Ausubel, et al. (1987, with periodic updates). Methods for chemical synthesis of nucleic acids are discussed, for example, in Beaucage and Carruthers, Tetra. Letts. 22:1859-1862, 1981, and Matteucci, et al., J. Am. Chem. Soc. 103:3185 (1981). Chemical synthesis of nucleic acids can be performed, for example, on commercial automated oligonucleotide synthesizers.

It has been found that not all hTIP39 encoding nucleic acid molecules are expressed in all neural tissues or in all portions of the brain. Thus, in order to isolate cDNA encoding the invention peptide or its splice variant, it is preferable to screen libraries prepared from different neuronal or neural tissues.

As used herein, a “splice variant” refers to variant invention peptide(s)-encoding nucleic acid(s) produced by differential processing of primary transcript(s) of genomic DNA, resulting in the production of more than one type of mRNA. cDNA derived from differentially processed primary transcript will encode the invention peptide(s) that have regions of complete amino acid identity and regions having different amino acid sequences. Thus, the same genomic sequence can lead to the production of multiple, related mRNAs and proteins. Both the resulting mRNAs and proteins are referred to herein as “splice variants”.

As used herein, a nucleic acid “probe” is single-stranded DNA or RNA, or analog thereof, that has a sequence of nucleotides that includes at least 14, preferably at least 20, more preferably at least 50, contiguous bases that are the same as or the complement of any 14 or more contiguous bases set forth in any of SEQ ID NO:1. In addition, the entire cDNA encoding region of the invention polypeptide, or the entire sequence corresponding to SEQ ID NO:1 may be used as a probe.

Presently preferred probe-based screening conditions comprise a temperature of about 37° C., a formamide concentration of about 20%, and a salt concentration of about 5× standard saline citrate (SSC; 20× SSC contains 3M sodium chloride, 0.3M sodium citrate, pH 7.0). Such conditions will allow the identification of sequences which have a substantial degree of similarity with the probe sequence, without requiring perfect homology.

Preferably, hybridization conditions will be selected which allow the identification of sequences having at least 70% homology with the probe, while discriminating against sequences which have a lower degree of homology with the probe. As a result, nucleic acids having substantially the same nucleotide sequence as the sequence of nucleotides set forth in SEQ ID NO:1 are obtained.

After screening the library, positive clones are identified by detecting a hybridization signal; the identified clones are characterized by restriction enzyme mapping and/or DNA sequence analysis, and then examined, by comparison with the sequences set forth herein, to ascertain whether they include DNA encoding the entire invention peptide. If the selected clones are incomplete, they may be used to rescreen the same or a different library to obtain overlapping clones. If desired, the library can be rescreened with positive clones until overlapping clones that encode an entire invention peptide are obtained. If the library is a cDNA library, then the overlapping clones will include an open reading frame. If the library is genomic, then the overlapping clones may include exons and introns. In both instances, complete clones may be identified by comparison with the DNA and encoded proteins provided herein.

Thus, the nucleic acid probes are useful for various applications. On the one hand, they may be used as PCR primers for amplification of nucleic acid molecules according to the invention. On the other hand, they can be useful tools for the detection of the expression of molecules according to the invention in target tissues, for example, by in-situ hybridization or Northern-Blot hybridization.

The invention probes may be labeled by methods well-known in the art, as described hereinafter, and used in various diagnostic kits.

A “label” refers to a compound or composition that facilitates detection of a compound or composition with which it is specifically associated, which can include conferring a property that makes the labeled compound or composition able to bind specifically to another molecule. “Labeled” refers to a compound or composition that is specifically associated, typically by covalent bonding but non-covalent interactions can also be employed to label a compound or composition, with a label. Thus, a label may be detectable directly, i.e., the label can be a radioisotope (e.g., ³H, ¹⁴C, ³²P, ³⁵S, ¹²⁵I, ¹³¹I or a fluorescent or phosphorescent molecule (e.g., FITC, rhodamine, lanthanide phosphors), or indirectly, i.e., by enzymatic activity (e.g., horseradish peroxidase, beta-galactosidase, luciferase, alkaline phosphatase) or by its ability to bind to another molecule (e.g., streptavidin, biotin, an antigen, epitope, or antibody). Incorporation of a label can be achieved by a variety of means, ie., by use of radiolabeled or biotinylated nucleotides in polymerase-mediated primer extension reactions, epitope-tagging via recombinant expression or synthetic means, or binding to an antibody.

Labels can be attached directly or via spacer arms of various lengths, i.e., to reduce steric hindrance. Any of a wide variety of labeled reagents can be used for purposes of the present invention. For instance, one can use one or more labeled nucleoside triphosphates, primers, linkers, or probes. A description of immunofluorescent analytic techniques is found in DeLuca, “Immunofluorescence Analysis”, in Antibody As a Tool, Marchalonis, et al., eds., John Wiley & Sons, Ltd., pp. 189-231 (1982), which is incorporated herein by reference.

The term label can also refer to a “tag”, which can bind specifically to a labeled molecule. For instance, one can use biotin as a tag and then use avidinylated or streptavidinylated horseradish peroxidase (HRP) to bind to the tag, and then use a chromogenic substrate (e.g., tetramethylbenzamine) to detect the presence of HRP. In a similar fashion, the tag can be an epitope or antigen (e.g., digoxigenin), and an enzymatically, fluorescently, or radioactively labeled antibody can be used to bind to the tag.

Use of the terms “isolated” and/or “purified” in the present specification and claims as a modifier of DNA, RNA, polypeptides or proteins means that the DNA, RNA, polypeptides or proteins so designated have been produced in such form by the hand of man, and thus are separated from their native in vivo cellular environment. As a result of this human intervention, the recombinant DNAs, RNAs, polypeptides and proteins of the invention are useful in ways described herein that the DNAs, RNAs, polypeptides or proteins as they naturally occur are not.

Similarly, as used herein, “recombinant” as a modifier of DNA, RNA, polypeptides or proteins means that the DNA, RNA, polypeptides or proteins so designated have been prepared by the efforts of human beings, e.g., by cloning, recombinant expression, and the like. Thus as used herein, recombinant proteins, for example, refers to proteins produced by a recombinant host, expressing DNAs which have been added to that host through the efforts of human beings.

As used herein, “mammalian” refers to the variety of species from which the invention TIP39 protein is derived, e.g., human, rat, mouse, rabbit, monkey, baboon, chicken, bovine, porcine, ovine, canine, feline, and the like. A preferred TIP39 protein herein, is human TIP39.

In one embodiment of the present invention, cDNAs encoding the invention peptide disclosed herein include substantially the same nucleotide sequence as set forth in SEQ ID NO:1. Preferred cDNA molecules encoding the invention proteins include the same nucleotide sequence as that set forth in SEQ ID NO:1.

Another embodiment of the invention contemplates nucleic acid(s) having substantially the same nucleotide sequence as the reference nucleotide sequence that encodes substantially the same amino acid sequence as that set forth in SEQ ID NO:2 or 14, preferably SEQ ID NO: 14 as it relates to the polypeptide corresponding to that of a homosapien.

In defining nucleic acid sequences, all subject nucleic acid sequences capable of encoding substantially similar amino acid sequences are considered substantially similar or are considered as comprising substantially identical sequences of nucleotides to the reference nucleic acid sequence, i.e., human TIP39 encoding sequence.

In practice, the term “substantially the same sequence” means that DNA or RNA encoding two proteins hybridize under moderately stringent conditions and encode proteins that have the same sequence of amino acids or have changes in sequence that do not alter their structure or function.

Nucleotide sequence “similarity” is a measure of the degree to which two polynucleotide sequences have identical nucleotide bases at corresponding positions in their sequence when optimally aligned (with appropriate nucleotide insertions or deletions). Sequence similarity or percent similarity can be determined, for example, by comparing sequence information using sequence analysis software such as the the GAP computer program, version 6.0, available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math. 2:482, 1981).

As used herein, “substantially identical sequences of nucleotides” share at least about 90% identity, and substantially identical amino acid sequences share more than 95% amino acid identity. It is recognized, however, that proteins (and DNA or mRNA encoding such proteins) containing less than the above-described level of homology arising as splice variants or that are modified by conservative amino acid substitutions (or substitution of degenerate codons) are contemplated to be within the scope of the present invention.

The present invention also encompasses nucleic acids which differ from the nucleic acids shown in SEQ ID NO:1, but which have the same phenotype. Phenotypically similar nucleic acids are also referred to as “functionally equivalent nucleic acids”.

As used herein, the phrase “functionally equivalent nucleic acids” encompasses nucleic acids characterized by slight and non-consequential sequence variations that will function in substantially the same manner to produce the same protein product(s) as the nucleic acids disclosed herein.

Functionally equivalent sequences will function in substantially the same manner to produce substantially the same compositions as the nucleic acid and amino acid compositions disclosed and claimed herein. In particular, functionally equivalent DNAs encode proteins that are the same as those disclosed herein or that have conservative amino acid variations, such as substitution of a non-polar residue for another non-polar residue or a charged residue for a similarly charged residue. These changes include those recognized by those of skill in the art as those that do not substantially alter the tertiary structure of the protein.

In particular, functionally equivalent nucleic acids encode polypeptides that are the same as those disclosed herein or that have conservative amino acid variations, or that are substantially similar to one having the amino acid sequence as set forth in SEQ ID NO:2 or 14, preferably SEQ ID NO:14.

For example, conservative variations include substitution of a non-polar residue with another non-polar residue, or substitution of a charged residue with a similarly charged residue. These variations include those recognized by skilled artisans as those that do not substantially alter the tertiary structure of the protein.

Further provided are nucleic acids encoding the invention polypeptides that, by virtue of the degeneracy of the genetic code, do not necessarily hybridize to the invention nucleic acids under specified hybridization conditions. Preferred nucleic acids encoding the invention polypeptide are comprised of nucleotides that encode substantially the same amino acid sequence set forth in SEQ ID NO: 2 or 14, preferably SEQ ID NO:14.

Thus, an exemplary nucleic acid encoding an invention polypeptide may be selected from:

(a) DNA encoding the amino acid sequence set forth in SEQ ID NO: 2 or 14, preferably the latter.

(b) DNA that hybridizes to the DNA of (a) under moderately stringent conditions, wherein said DNA encodes biologically active human TIP39; or

(c) DNA degenerate with respect to either (a) or (b) above, wherein said DNA encodes biologically active human TIP39.

As used herein, the term “degenerate” refers to codons that differ in at least one nucleotide from SEQ ID NO:1, but encode the same amino acids as that set forth in SEQ ID NO: 2 or 14, preferably SEQ ID NO: 14. For example, codons specified by the triplets “UCU”, “UCC”, “UCA”, and “UCG” are degenerate with respect to each other since all four of these codons encode the amino acid serine.

As used herein, the “amino acid sequence” of SEQ ID NO: 1 or 2 refers to the deduced amino acid sequence set forth in SEQ ID NO: 1 or the amino acid sequence set forth in SEQ ID NO: 2 corresponding to humans. Each of the amino acid sequences are those of the immature protein, i.e., prior to post-translational modification. Likewise, SEQ ID NO: 14 refers to the mature polypeptide.

Hybridization refers to the binding of complementary strands of nucleic acid (i.e., sense:antisense strands or probe:target-DNA) to each other through hydrogen bonds, similar to the bonds that naturally occur in chromosomal DNA. Stringency levels used to hybridize a given probe with target-DNA can be readily varied by those of skill in the art.

The phrase “stringent hybridization” is used herein to refer to conditions under which polynucleic acid hybrids are stable. As known to those of skill in the art, the stability of hybrids is reflected in the melting temperature (Tₘ) of the hybrids. Tₘ can be approximated by the formula:

81.5° C.−16.6(log₁₀[Na⁺])+0.41(% G+C)−600/1,

where I is the length of the hybrids in nucleotides. Tₘ decreases approximately 1°-1.5° C. with every 1% decrease in sequence homology. In general, the stability of a hybrid is a function of sodium ion concentration and temperature. Typically, the hybridization reaction is performed under conditions of lower stringency, followed by washes of varying, but higher, stringency. Reference to hybridization stringency relates to such washing conditions.

As used herein, the phrase “moderately stringent hybridization” refers to conditions that permit target-DNA to bind a complementary nucleic acid that has about 60% identity, preferably about 75% identity, more preferably about 85% identity to the target DNA; with greater than about 90% identity to target-DNA being especially preferred. Preferably, moderately stringent conditions are conditions equivalent to hybridization in 50% formamide, 5× Denhart's solution, 5× SSPE, 0.2% SDS at 42° C., followed by washing in 0.2× SSPE, 0.2% SDS, at 65° C.

The phrase “high stringency hybridization” refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in 50% formamide, 5× Denhart's solution, 5× SSPE, 0.2% SDS at 42° C., followed by washing in 0.1× SSPE, and 0.1% SDS at 65° C.

The phrase “low stringency hybridization” refers to conditions equivalent to hybridization in 10% formamide, 5× Denhart's solution, 6× SSPE, 0.2% SDS at 42° C., followed by washing in 1× SSPE, 0.2% SDS, at 50° C.

Denhardt's solution and SSPE (see, e.g., Sambrook, Fritsch, and Maniatis, in: Molecular Cloning, A Laboratory Manual, Cold Spring Harbor Laboratory Press, 1989) are well known to those of skill in the art as are other suitable hybridization buffers. For example, SSPE is pH 7.4 phosphate-buffered 0. 18M NaCl. SSPE can be prepared, for example, as a 20× stock solution by dissolving 175.3 g of NaCl, 27.6 g of NaH₂PO₄ and 7.4 g EDTA in 800 ml of water, adjusting the pH to 7.4, and then adding water to 1 liter. Denhardt's solution (see, Denhardt (1966) Biochem. Biophys. Res. Commun. 23:641) can be prepared, for example, as a 50× stock solution by mixing 5 g Ficoll (Type 400, Pharmacia LKB Biotechnology, INC., Piscataway N.J.), 5 g of polyvinylpyrrolidone, and 5 g bovine serum albumin (Fraction V; Sigma, St. Louis Mo.), and then adding water to 500 ml and filtering to remove particulate matter.

Preferred nucleic acids encoding the invention polypeptide(s) hybridize under moderately stringent, preferably high stringency, conditions to substantially the entire sequence, or substantial portions (i.e., typically at least 15-30 nucleotides) of the nucleic acid sequence set forth in SEQ ID NO:1.

The invention nucleic acids can be produced by a variety of methods well-known in the art, e.g., the methods described herein, employing PCR amplification using oligonucleotide primers from various regions of SEQ ID NO:1 and the like.

As used herein, “expression” refers to the process by which polynucleic acids are transcribed into mRNA and translated into peptides, polypeptides, or proteins. If the polynucleic acid is derived from genomic DNA, expression may, if an appropriate eukaryotic host cell or organism is selected, include splicing of the mRNA.

An example of the means for preparing the invention polypeptide(s) is to express nucleic acids encoding the invention polypeptide in a suitable host cell, such as a bacterial cell, a yeast cell, an amphibian cell (i.e., oocyte), or a mammalian cell, using methods well known in the art, and recovering the expressed polypeptide, again using well-known methods. Invention polypeptides can be isolated directly from cells that have been transformed with expression vectors comprising nucleic acid encoding the invention peptides or fragments/portions thereof.

Incorporation of cloned DNA into a suitable expression vector, transfection of eukaryotic cells with a plasmid vector or a combination of plasmid vectors, each encoding one or more distinct genes or with linear DNA, and selection of transfected cells are well known in the art (see, e.g., Sambrook, et al. (1989) Molecular Cloning: A Laboratory Manual, Second Edition, Cold Spring Harbor Laboratory Press). Suitable means for introducing (transducing) expression vectors containing invention nucleic acid constructs into host cells to produce transduced recombinant cells (i.e., cells containing recombinant heterologous nucleic acid) are well-known in the art (see, for review, Friedmann, 1989, Science, 244:1275-1281; Mulligan, 1993, Science, 260:926-932, each of which are incorporated herein by reference in their entirety).

Exemplary methods of transduction include, e.g., infection employing viral vectors (see, e.g., U.S. Pat. Nos. 4,405,712 and 4,650,764), calcium phosphate transfection (U.S. Pat. Nos. 4,399,216 and 4,634,665), dextran sulfate transfection, electroporation, lipofection (see, e.g., U.S. Pat. Nos. 4,394,448 and 4,619,794), cytofection, particle bead bombardment, and the like. The heterologous nucleic acid can optionally include sequences which allow for its extrachromosomal (i.e., episomal) maintenance, or the heterologous nucleic acid can be donor nucleic acid that integrates into the genome of the host. Recombinant cells can then be cultured under conditions whereby the invention peptide(s) encoded by the DNA is (are) expressed. Preferred cells include mammalian cells (e.g., HEK 293, CHO and Ltk⁻ cells), yeast cells (e.g., methylotrophic yeast cells, such as Pichia pastoris), bacterial cells (e.g., Escherichia coli), and the like.

Suitable expression vectors are well-known in the art, and include vectors capable of expressing DNA operatively linked to a regulatory sequence, such as a promoter region that is capable of regulating expression of such DNA. Thus, an expression vector refers to a recombinant DNA or RNA construct, such as a plasmid, a phage, recombinant virus or other vector that, upon introduction into an appropriate host cell, results in expression of the inserted DNA. Appropriate expression vectors are well known to those of skill in the art and include those that are replicable in eukaryotic cells and/or prokaryotic cells and those that remain episomal or those which integrate into the host cell genome.

Exemplary expression vectors for transformation of E. coli prokaryotic cells include the pET expression vectors (Novagen, Madison, Wis., see U.S. Pat. No. 4,952,496), e.g., pET11a, which contains the T7 promoter, T7 terminator, the inducible E. coli lac operator, and the lac repressor gene; and pET 12a-c, which contains the T7 promoter, T7 terminator, and the E. coli ompT secretion signal. Another such vector is the pIN-IIIompA2 (see Duffaud, et al., Meth. in Enzymology, 153:492-507, 1987), which contains the 1pp promoter, the lacUV5 promoter operator, the ompA secretion signal, and the lac repressor gene.

Exemplary eukaryotic expression vectors include eukaryotic cassettes, such as the pSV-2 gpt system (Mulligan, et al., 1979, Nature, 277:108-114); the Okayama-Berg system (Mol. Cell Biol., 2:161-170), and the expression cloning vector described by Genetics Institute (1985, Science, 228:810-815). Each of these plasmid vectors are capable of promoting expression of the invention chimeric protein of interest.

As used herein, “heterologous or foreign DNA and/or RNA” are used interchangeably and refer to DNA or RNA that does not occur naturally as part of the genome of the cell in which it is present or to DNA or RNA which is found in a location or locations in the genome that differ from that in which it occurs in nature. Typically, heterologous or foreign DNA and RNA refers to DNA or RNA that is not endogenous to the host cell and has been artificially introduced into the cell. Examples of heterologous DNA include DNA that encodes the invention peptides.

In preferred embodiments, DNA is ligated into a vector, and introduced into suitable host cells to produce transformed cell lines that express the invention peptide, or a fragment thereof. The resulting cell lines can then be produced in quantity for reproducible quantitative analysis of the effects of drugs on receptor function.

In other embodiments, mRNA may be produced by in vitro transcription of DNA encoding the invention peptide. This mRNA can then be injected into Xenopus oocytes where the RNA directs the synthesis of the invention peptide. Alternatively, the invention-encoding DNA can be directly injected into oocytes for expression of a functional invention peptide. The transfected mammalian cells or injected oocytes may then be used in the methods of drug screening provided herein.

Eukaryotic cells in which DNA or RNA may be introduced include any cells that are transfectable by such DNA or RNA or into which such DNA or RNA may be injected. Preferred cells are those that can be transiently or stably transfected and also express the DNA and RNA. Presently most preferred cells are those that can express recombinant or heterologous human TIP39 encoded by the heterologous DNA. Such cells may be identified empirically or selected from among those known to be readily transfected or injected.

Exemplary cells for introducing DNA include cells of mammalian origin (e.g., COS cells, mouse L cells, Chinese hamster ovary (CHO) cells, human embryonic kidney cells, African green monkey cells and other such cells known to those of skill in the art), amphibian cells (e.g., Xenopus laevis oocytes), yeast cells (e.g., Saccharomyces cerevisiae, Pichia pastoris), and the like. Exemplary cells for expressing injected RNA transcripts include Xenopus laevis oocytes. Cells that are preferred for transfection of DNA are known to those of skill in the art or may be empirically identified, and include HEK 293; Ltk⁻ cells; COS-7 cells ; and DG44 cells (dhrf⁻ CHO cells; see, e.g., Urlaub, et al. (1986) Cell. Molec. Geizet. 12:555). Other mammalian expression systems, including commercially available systems and other such systems known to those of skill in the art, for expression of DNA encoding the invention peptide provided herein are presently preferred.

Nucleic acid molecules may be stably incorporated into cells or may be transiently introduced using methods known in the art. Stably transfected mammalian cells may be prepared by transfecting cells with an expression vector having a selectable marker gene (such as, for example, the gene for thymidine kinase, dihydrofolate reductase, neomycin resistance, and the like), and growing the transfected cells under conditions selective for cells expressing the marker gene. To produce such cells, the cells should be transfected with a sufficient concentration of invention peptide-encoding nucleic acids to form the invention peptide(s) that are encoded by heterologous DNA. The precise amounts and ratios of DNA encoding the invention peptides may be empirically determined and optimized for a particular cells and assay conditions.

Heterologous DNA may be maintained in the cell as an episomal element or may be integrated into chromosomal DNA of the cell. The resulting recombinant cells may then be cultured or subcultured (or passaged, in the case of mammalian cells) from such a culture or a subculture thereof. Methods for transfection, injection and culturing recombinant cells are known to the skilled artisan. Similarly, the invention peptide(s) may be purified using protein purification methods known to those of skill in the art. For example, antibodies or other ligands that specifically bind to human TIP39 may be used for affinity purification of the invention peptide.

In accordance with the above, host cells are transfected with DNA encoding the invention peptide. Using methods such as northern blot or slot blot analysis, transfected cells that contain invention peptide encoding DNA or RNA can be selected. Transfected cells can also be analyzed to identify those that express the invention peptide. Analysis can be carried out, for example, by measuring the ability of cells to bind the PTH₂ receptor, or a PTH₂ receptor agonist, compared to the PTH₂ receptor binding ability of untransfected host cells or other suitable control cells, by electrophysiologically monitoring the currents through the cell membrane in response to invention peptide, and the like.

In particularly preferred aspects, eukaryotic cells which contain heterologous DNAs express such DNA and form recombinant invention peptide. In more preferred aspects, recombinant invention peptide activity is readily detectable because it is a type that is absent from the untransfected host cell.

As used herein, activity of the invention peptide refers to any activity characteristic of human TIP39. Such activity can typically be measured by one or more in vitro methods, and frequently corresponds to an in vivo activity of human TIP39. Such activity may be measured by any method known to those of skill in the art, such as, for example, assays that measure parathyroid hormone-2 receptor binding and cAMP levels.

The invention peptide, biologically active fragments, and functional equivalents thereof can also be produced by chemical synthesis. For example, synthetic polypeptides can be produced using Applied Biosystems, Inc. Model 430A or 431A automatic peptide synthesizer (Foster City, Calif.) employing the chemistry provided by the manufacturer.

The present invention also provides compositions containing an acceptable carrier and any of an isolated, purified invention polypeptide, an active fragment thereof, or a purified, mature protein and active fragments thereof, alone or in combination with each other. These polypeptides or proteins can be recombinantly derived, chemically synthesized or purified from native sources.

As used herein, the term “acceptable carrier” encompasses any of the standard pharmaceutical carriers, such as phosphate buffered saline solution, water and emulsions such as an oil/water or water/oil emulsion, and various types of wetting agents.

Also provided are antisense oligonucleotides having a nucleotide sequence capable of binding specifically with any portion of an mRNA that encodes the invention peptide so as to prevent translation of the mRNA. The antisense oligonucleotide may have a sequence capable of binding specifically with any portion of the sequence of the cDNA encoding the invention polypeptides.

In accordance with yet another embodiment of the present invention, there are provided anti-human TIP39 antibodies having specific affinity for the invention peptides. Active fragments of antibodies are encompassed within the definition of “antibody”.

Invention antibodies can be produced by methods known in the art using invention polypeptides, proteins or portions thereof as antigens. For example, polyclonal and monoclonal antibodies can be produced by methods well known in the art, as described, for example, in Harlow and Lane, Antibodies: A Laboratory Manual (Cold Spring Harbor Laboratory (1988)), which is incorporated herein by reference. Invention polypeptides can be used as immunogens in generating such antibodies. Alternatively, synthetic peptides can be prepared (using commercially available synthesizers) and used as immunogens. Amino acid sequences can be analyzed by methods well known in the art to determine whether they encode hydrophobic or hydrophilic domains of the corresponding polypeptide. Altered antibodies such as chimeric, humanized, CDR-grafted or bifunctional antibodies can also be produced by methods well known in the art. Such antibodies can also be produced by hybridoma, chemical synthesis or recombinant methods described, for example, in Sambrook, et al., supra., and Harlow and Lane, supra. Both anti-peptide and anti-fusion protein antibodies can be used. (see, for example, Bahouth, et al., Trends Pharmacol. Sci. 12:338 (1991); Ausubel, et al., Current Protocols in Molecular Biology (John Wiley and Sons, N.Y. (1989) which are incorporated herein by reference).

Antibody so produced can be used, inter alia, in diagnostic methods and systems to detect the level of the invention peptide(s) present in a mammalian, preferably human, body sample, such as tissue.

Such antibodies can also be used for the immunoaffinity or affinity chromatography purification of the invention polypeptide. In addition, methods are contemplated herein for detecting the presence of invention polypeptides on the surface of a cell comprising contacting the cell with an antibody that specifically binds to invention polypeptides, under conditions permitting binding of the antibody to the polypeptides, detecting the presence of the antibody bound to the cell, and thereby detecting the presence of invention polypeptides on the surface of the cell. With respect to the detection of such polypeptides, the antibodies can be used for in vitro diagnostic or in vivo imaging methods.

Immunological procedures useful for in vitro detection of invention polypeptides in a sample include immunoassays that employ a detectable antibody. Such immunoassays include, for example, ELISA, Pandex microfluorimetric assay, agglutination assays, flow cytometry, serum diagnostic assays and immunohistochemical staining procedures, which are well known in the art. An antibody can be made detectable by various means well known in the art. For example, a detectable marker can be directly or indirectly attached to the antibody. Useful markers include, for example, radionucleotides, enzymes, fluorogens, chromogens and chemiluminescent labels.

The above referenced anti-human TIP39 antibodies can also be used to modulate the activity of the invention peptide in living animals, in humans, or in biological tissues isolated therefrom. Accordingly, compositions comprising a carrier and an amount of an antibody having specificity for the invention peptide effective to block naturally occurring TIP39 or other ligands from binding to PTH₂ receptor are contemplated herein. For example, a monoclonal antibody directed to an epitope of the invention peptide molecule and having an amino acid sequence substantially the same as an amino acid sequence as shown in SEQ ID NO: may be useful for blocking binding of the invention polypeptide to human PTH₂ receptor.

“Immunologically active fragment(s)” of the invention peptides are also embraced by the invention. Such fragments are those proteins that are capable of raising human TIP39-specific antibodies in a target immune system (e.g., murine or rabbit) or of competing with human TIP39 for binding to hTIP39-specific antibodies, and is thus useful in immunoassays for the presence of human TIP39 peptides in a biological sample. Such immunologically active fragments typically have a minimum size of 8 to 11 consecutive amino acids of a native human TIP39 peptide.

The present invention further provides transgenic non-human mammals that are capable of expressing exogenous nucleic acids encoding the invention peptides. As employed herein, the phrase “exogenous nucleic acid” refers to nucleic acid sequence which is not native to the host, or which is present in the host in other than its native environment (e.g., as part of a genetically engineered DNA construct). A transgenic mouse expressing exogenous invention nucleic acid encoding the invention peptide is particularly preferred.

Animal model systems which elucidate the physiological and behavioral roles of the invention peptides are also contemplated, and may be produced by creating transgenic animals in which the expression of the invention peptide is altered using a variety of techniques. Examples of such techniques include the insertion of normal or mutant versions of nucleic acids encoding the invention polypeptide by microinjection, retroviral infection or other means well known to those skilled in the art, into appropriate fertilized embryos to produce a transgenic animal (Hogan, et al., Manipulating the Mouse Embryo: A Laboratory Manual (Cold Spring Harbor Laboratory, (1986)).

Invention nucleic acids, oligonucleotides (including antisense), vectors containing same, transformed host cells, polypeptides and combinations thereof, as well as antibodies of the present invention, can be used to screen compounds in vitro to determine whether a compound functions as a potential agonist or antagonist to invention peptides.

These in vitro screening assays provide information regarding the function and activity of invention peptides, which can lead to the identification and design of compounds that are capable of specific interaction with native human TIP39 or the human PTH₂ receptor.

Accordingly, a method for identifying compounds, which bind to the invention peptide(s) are also contemplated by the present invention. The invention peptide may be employed in a competitive binding assay. Such an assay can accommodate the rapid screening of a large number of compounds to determine which compounds, if any, are capable of binding to invention peptide. Subsequently, more detailed assays can be carried out with those compounds found to bind, to further determine whether such compounds act as modulators, agonists or antagonists of invention peptide.

In accordance with another embodiment of the present invention, transformed host cells that recombinantly express the PTH₂ receptor can be contacted with a test compound, and the modulating effect(s) thereof can then be evaluated by comparing the invention peptide-mediated response (e.g., via measurement of second messenger activity/cAMP activity) in the presence and absence of the test compound, or by comparing the response of test cells or control cells, i.e., cells that do not express the invention peptides to the presence of the compound.

As used herein, a compound or a signal that “modulates the activity” of invention peptide refers to a compound or a signal that alters the activity of invention peptide so that the activity of the invention peptide is different in the presence of the compound or signal than in the absence of the compound or signal. In particular, such compounds or signals include agonists and antagonists. Such activity is generally detected by measuring cAMP levels.

The term “agonist” refers to a substance or signal, such as the invention peptide, that activates receptor function; and the term “antagonist” refers to a substance that interferes with receptor function. Typically, the effect of an antagonist is observed as a blocking of activation by an agonist. Antagonists include competitive and non-competitive antagonists. A competitive antagonist (or competitive blocker) interacts with or near the site specific for the agonist (e.g., ligand or neurotransmitter) for the same or closely situated site. A non-competitive antagonist or blocker inactivates the functioning of the receptor by interacting with a site other than the site that interacts with the agonist.

As understood by those of skill in the art, assay methods for identifying compounds that modulate invention peptide activity generally require comparison to a control. One type of a “control” is a cell or culture that is treated substantially the same as the test cell or test culture exposed to the compound, with the distinction that the “control” cell or culture is not exposed to the compound. For example, in methods that use voltage clamp electrophysiological procedures, the same cell can be tested in the presence or absence of compound, by merely changing the external solution bathing the cell. Another type of “control” cell or culture may be a cell or culture that is identical to the transfected cells, with the exception that the “control” cell or culture do not express the invention peptide, but like the transfected cell expresses a functional human PTH2 receptor. Accordingly, the response of the transfected cell to compound is compared to the response (or lack thereof) of the “control” cell or culture to the same compound under the same reaction conditions.

In yet another embodiment of the present invention, the activation of invention peptides can be modulated by contacting the polypeptides with an effective amount of at least one compound identified by the above-described bioassays.

An alternative method contemplates contacting a cell expressing a PTH₂ receptor with a test compound in the presence of the invention peptide, and determining the effect of the test compound by measuring level of cAMP as a measure of the modulating effect of the test compound on PTH₂ receptor activity, wherein an increase in cAMP levels is indicative of the modulating effects of the test compound on the PTH₂ receptor (agonist), i.e., opening of the PTH₂ receptor, while a decrease reflects the opposite (antagonist).

In accordance with another embodiment of the present invention, there are provided methods for diagnosing disease states characterized by abnormal signal transduction. For example, a sample can be obtained from a patient believed to be suffering from a pathological disorder characterized by dysfunctional signal transduction, and contacted with a nucleic acid probe having a sequence of nucleotides that are substantially homologous to the nucleotide sequence set forth in one of SEQ ID NO:1. Binding of the probe to any complimentary mRNA present in the sample can be determined and is indicative of the regression, progression or onset of such a pathological disorder in the patient.

Alternatively, the patient sample can be contacted with a detectable probe that is specific for the gene product of the invention nucleic acid molecule, under conditions favoring the formation of a probe/gene product complex. The presence of the complex is indicative of the regression, progression or onset of said pathological disorder in the patient.

In accordance with another embodiment of the present invention, there are provided diagnostic systems, preferably in kit form, comprising at least one invention nucleic acid in a suitable packaging material. The diagnostic nucleic acids are derived from the invention peptide-encoding nucleic acids described herein. In one embodiment, for example, the diagnostic nucleic acids are derived from SEQ ID NO:1. Invention diagnostic systems are useful for assaying for the presence or absence of nucleic acid encoding the invention peptide in either genomic DNA or in transcribed nucleic acid (such as mRNA or cDNA) encoding the invention peptide.

A suitable diagnostic system includes at least one invention nucleic acid, preferably two or more invention nucleic acids, as a separately packaged chemical reagent(s) in an amount sufficient for at least one assay. Instructions for use of the packaged reagent are also typically included. Those of skill in the art can readily incorporate invention nucleic probes and/or primers into kit form in combination with appropriate buffers and solutions for the practice of the invention methods as described herein.

“Treatment” refers to both therapeutic treatment and prophylactic or preventative measures. Those in need of treatment include those already with the disorder as well as those prone to have the disorder or those in which the disorder is to be prevented.

A “disorder” is any condition that would benefit from treatment with the invention peptide of the invention. This includes chronic and acute disorders or diseases including those pathological conditions which predispose the mammal to the disorder in question. Disorders include, but are not limited to, those of the cardiovascular system, the nervous system and those involving pain perception.

As used herein, “functional” with respect to a recombinant or heterologous human TIP39 means that the peptide exhibits an activity attending native human TIP39 as assessed by any in vitro or in vivo assay disclosed herein or known to those of skill in the art. Possession of any such activity that may be assessed by any method known to those of skill in the art and provided herein is sufficient to designate a peptide as functional. Such activity may be detected as noted supra.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

### EXAMPLE 1

### Isolation of DNA Encoding Human tuberoinfundibular peptide of 39 residues

I. DNA Encoding a human tuberoinfundibular peptide of 39 residues

A. Cloning of human TIP39 cDNA

A cDNA fragment specific to human TIP39 was generated by PCR amplification of human hypothalamus cDNA. The following degenerate oligonucleotide primers were utilized to generate a fragment for plasmid subcloning:

| | | |
|---|---|---|
| Hw37 | [TIGCIGA(T/C)GA(T/C)GCIGCITTCCG] and | (SEQ ID NO:4) |
| Hw39 | [TCIA(A/G)IACIA(A/G)IA(A/G)IA(A/G)(C/T)TTGTGCAT]. | (SEQ ID NO:5) |

The resulting 98 bp fragment was subcloned into the PCRII vector as described by the manufacturer and sequenced. Sequence analysis indicated that the fragment encodes a peptide that aligns with bovine TIP39 peptide between positions 5-36. The sequence information obtained from this clone was utilized to design the following oligonucleotide primer pair, which should yield a PCR fragment of approximately 70 bp:

| | | |
|---|---|---|
| Hw60 | (TGCATGTACGAGTTCAGCCAGTGG) and | (SEQ ID NO:6) |
| KB01 | (CTTCCGGGAGCGCGCGCGGTTG). | (SEQ ID NO:7) |

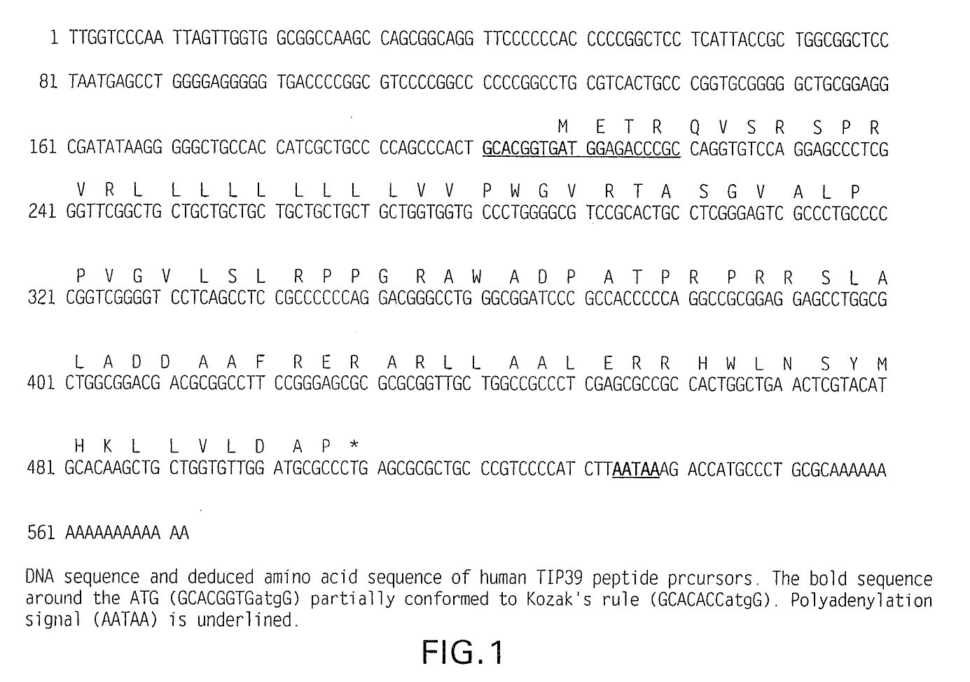

The Hw60 and KB01 primers were used to screen a human fetal brain stem cDNA library (Incyte/Genomic Systems). Two identical clones were identified and sequenced. The DNA sequence and deduced amino acid sequence of the precursor of TIP39 is shown in FIG. 1.

B. Cloning of mouse TIP39 cDNA

To clone mouse TIP39, a genomic clone homologous to human TIP39 was obtained and the region that contains TIP39 was sequenced. Primers derived from mouse genomic sequence were used to screen a mouse brain stem cDNA library (Incyte/Genomic Systems).

| | | |
|---|---|---|
| HS05: | CTAGCTGACGACGCGGCCTTC | (SEQ ID NO:8) |
| HS07: | GGGCGCGTCCAGTAGCAACAGC | (SEQ ID NO:9) |

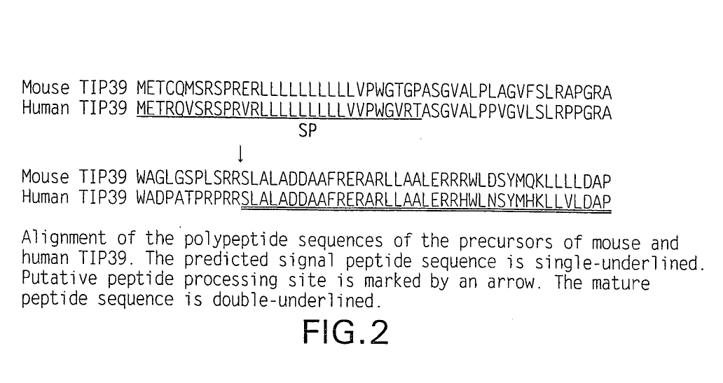

Two identical clones were obtained and sequenced. Amino acid sequence of the precursor of mouse TIP39 is homologous to Human TIP39. The predicted sequences of the prepolypeptide and mature peptide (double-underlined) are shown in FIG. 2.

C. PCR amplification of rat TIP39.

Rat TIP39 gene was PCR amplified from rat brain cDNA using primers derived from mouse cDNA.

| | | |
|---|---|---|
| HS16: | CTTGGGTAGCCCCCTGTCTCGG | (SEQ ID NO:10) |
| HS07: | GGGCGCGTCCAGTAGCAACAGC | (DEQ ID NO:9) |

The alignment of mature human, bovine, rat and mouse TIP39 peptide sequence is shown in FIG. 3.

Recombinant cell lines generated by transfection with DNA encoding human TIP39 can be further characterized using one or more of the following methods.

(a) Northern or slot blot analysis for expression of human tuberoinfundibular peptide of 39 residues encoding messages

(b) Immunostaining with a TIP39 specific antibody or measuring cAMP production.

### EXAMPLE 2

### Functional assays involving TIP39 in activation of its receptor

1. Establishment of cell lines that stably express rat and human PTH₂R.

HEK293 cells were transfected with rat and human PTH₂R and cell lines that stably express the receptors were established and evaluated. One cell line was selected for each rat and human PTH₂R and used for all the functional assays. Specifically, HEK293 cells were transfected with pCDNA3.1/V5-His-RatPTH₂R plasmid DNA and pCDNA3.1-E/Uni-lacZ-HumanPTH₂R plasmid DNA using FuGENE 6 transfection reagent (Roche Molecular Biochemicals). Three days after transfection, cells were put under selection with 800 μg/ml geneticin (G418, Gibco BRL). After three weeks of selection, single colonies were cloned using cloning cylinders. Cloned colonies were scaled up to T25 flasks. More than 10 cell lines for each plasmid were tested for function in the cAMP SPA assay. One cell line per plasmid was chosen for further characterization based on the cyclase assay results.

2. cAMP SPA Assay for PTH₂R activation

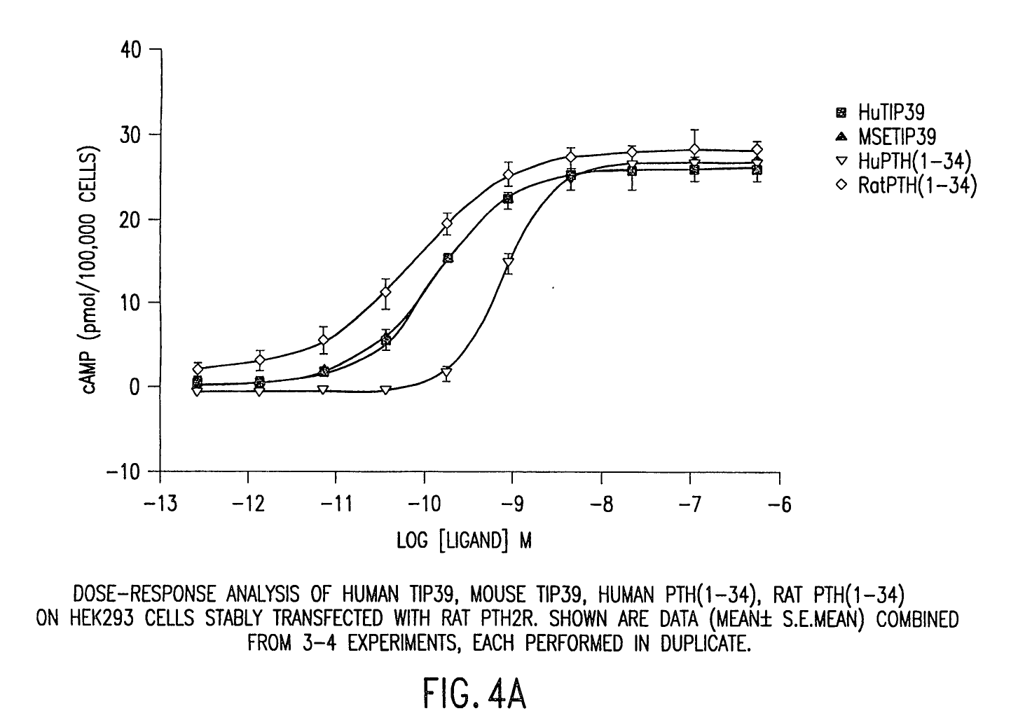

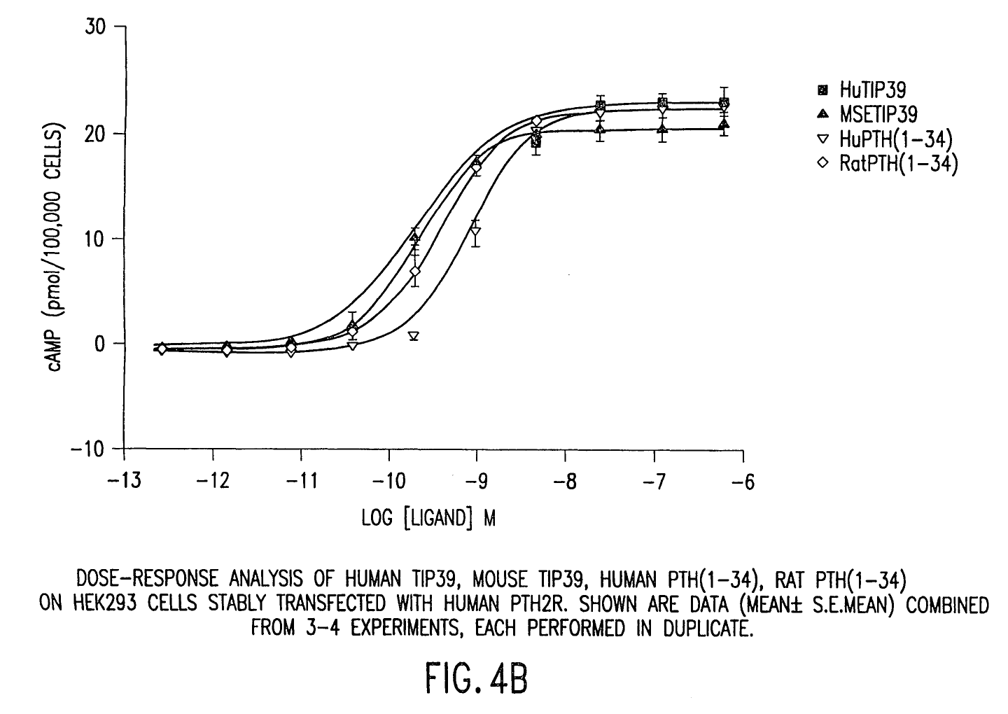

HEK293PTH₂R cells were seeded in a 96-well poly-D-lysine coated plate at 100,000 cells per well and cultured overnight. After washing with 200 μl of PBS and treating with 100 μl of 300 nM IBMX in assay medium (MEM without phenol red and FBS) at 37° C. for 10 min. The assay medium was then aspirated and the cells were washed with PBS. The cellular of cAMP was measured using cAMP SPA Direct Screening (Amersham/Pharmacia RPA559). Each concentration of ligand was repeated as triplicate. The dose-response curves of the ligands on HEK293 cells stably transfected with rat and human PTH₂R are shown in FIG. 4A and FIG. 4B, respectively.

Results: In order to test cAMP response in HEK293 cells stably transfected with rat and human PTH₂R, the level of cAMP upon stimulation with an agonist peptide in HEK 293 cells expressing rat PTH₂R was measured. Positive responses were observed when cells expressing the rat PTH cells were stimulated with Human TIP39, mouse TIIP39, human PTH (1-34) and rat PTH(1-34) i.e., an increase in the level of cAMP to about the same maximal level ( up to 29 pmol/100,000 cells). The increase in cAMP was dose-dependent and is depicted in FIG. 4A. The logEC50 for HumanTIP39, Mouse TIP39, human PTH(1-34) and rat PTH(1-34) are −9.7±0.2, −9.9±0.1.−9.3±0.2 and −10.3±0.5, respectively (mean±S. D., n=3-7). The response with human PTH(1-34) appears to be slightly less potent as shown in FIG. 4A.

A similar increase in cAMP level upon stimulation with these peptides in HEK293 cells that expresses human PTH₂R was also observed. The maximal response reached 25 pmol/100,000 cells. The potency of these peptides appeared to be similar to that detected in cells expressing rat PTH₂R. logEC50 for Human TIP39, mouse TIP39, Human PTH(1-34) and rat PTH(1-34) in HEK293 cells that expressed human PTH₂R was recorded as follows: −9.7±0.2,−9.7±0.3, −9.1±0.2 and −9.8±0.4, respectively(mean+S. D, n=4-7). Again, human PTH(1-34) appeared to be slightly less potent than the other peptides (FIG. 4B).

3. Measurement of [Ca²⁺]i using the FLIPR

HEK293 cells were seeded into black walnut clear-base 96 well plates (BD Biocoat) coated with poly-D-lysine at a density of 100,000 cells per well in MEM, and cultured overnight. The cells were then washed two times with 1×HBSS buffer (1× Hank's balanced Salt Solution (Life Technologies), 20 mM HEPES, 2 mM Calcium Chloride, 0.12 mM probenecid (Sigma, (710mg/5ml 1N NaOH)), then loaded with loading buffer (HBSS+10%FBS+4uM Fluo-3AM cell permeant Molecular Probes) at 37° C. for 60 min. The cells were washed two times with 1×HBSS buffer. The plates were then placed into a FLIPR (Molecular Devices) to monitor cell fluorescence (ex=488 nM, EM=540 nM) before and after the addition of various agonists for 3 minutes. Responses were measured as peak fluorescence intensity (FI) minus basal FI, and where appropriate were expressed as a percentage of a maximum human TIP39-induced response.

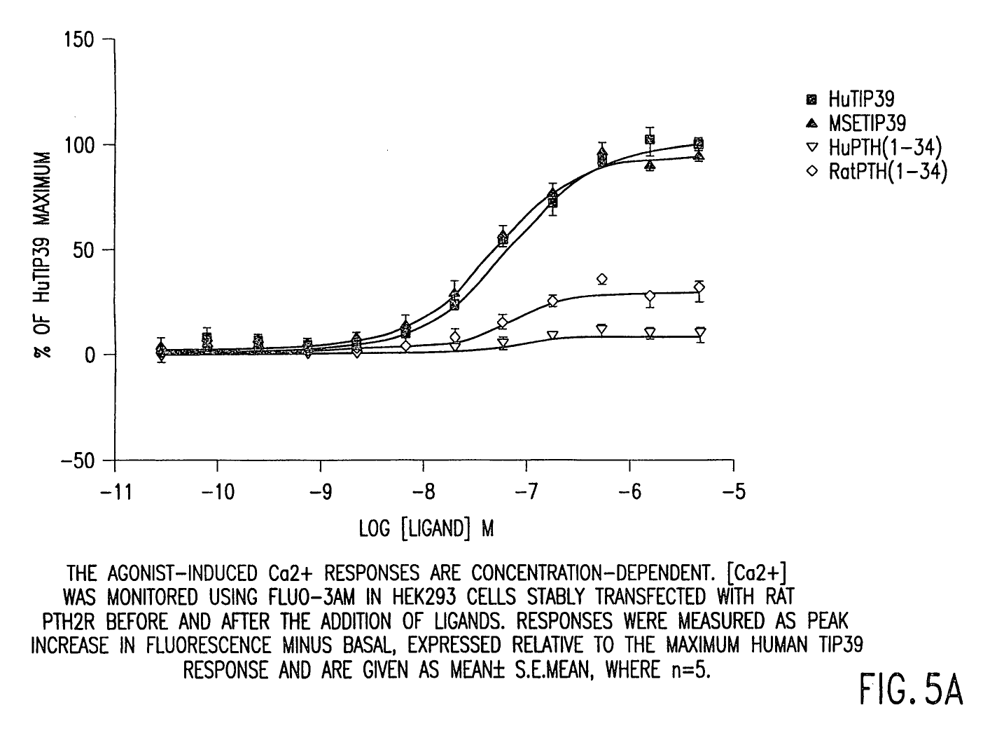

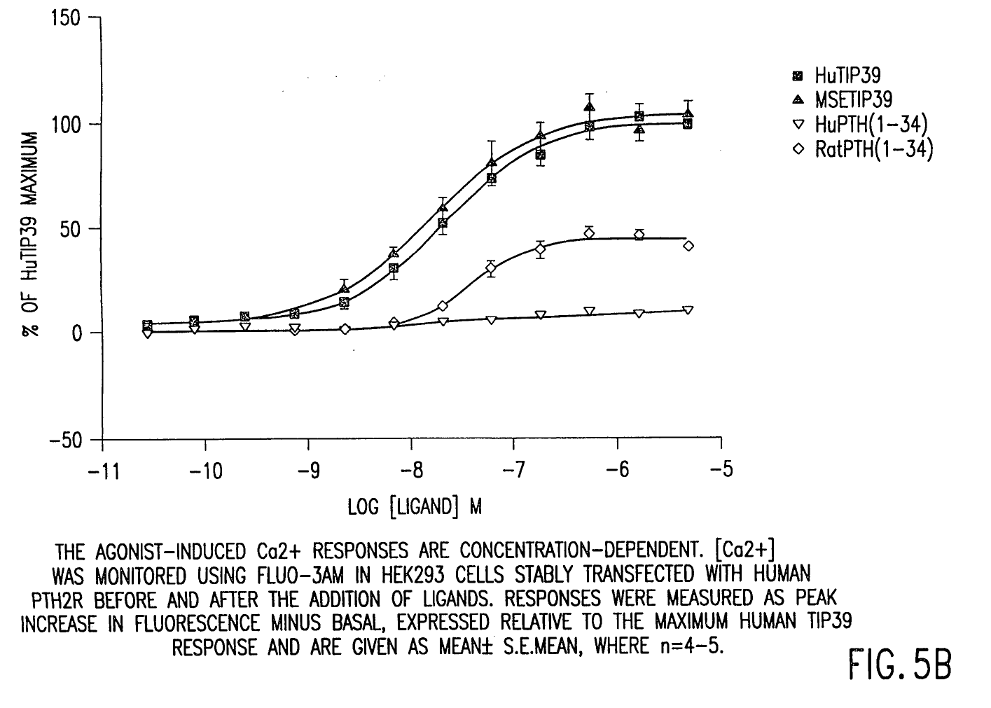

Curve-fitting and parameter estimation were carried out using Graph Pad Prism, 3.00 (GraphPad Software Inc., California, U.S.A.). The dose-response curves for various ligand on the rat and human PTH₂R cell lines are shown in FIG. 5A and FIG. 5B, respectively.

Results: Overall, human TIP39 caused an increase in intracellular calcium concentration. Importantly, human TIP39 caused an increase in [Ca²⁺]i in HEK293 that expresses rat PTH₂R, which was typified by initially rapid onset (peak˜20S), followed by a rapidly declining secondary phase, returning to baseline level within 80S (data not shown). Mouse TIP39 also increased [Ca²⁺]i with similar kinetics.

When measuring the peak fluorescence change upon stimulation with human TIP39, mouse TIP39, Human PTH(1-34) and rat PTH(1-34), - Human and Mouse TIP39 were observed to be agonist compared to rat PTH and human PTH. Maximum response from rat PTH was ca 30% compared to that of human TIP39 on rat PTH₂R expressing cells. Human PTH(1-34) was found to elicit little response if any.

All agonist-induced responses were concentration-dependent (FIG. 5A). The logEC50 for human TIP39, mouse TIP39, and rat PTH(1-34) on HEK293 cells that express rat PTH₂R were observed to be −7.2±0.1, −7.3±0.1 and −7.4±0.1, respectively (mean±S.D, n=4-5). A similar rapid rise in intracellular calcium in HEK293 cells that expresses human PTH₂R was also observed. The data suggest that Human TIP39 and mouse TIP39 are full agonists in comparison to the rat and human PTH. Human PTH(1-34) appeared to elicit little response. Maximum response elicited by rat PTH(1-34) was 42±2% compared to that elicited by human TIP39. The logEC50 for human TIP39, mouse TIP39, and rat PTH(1-34) are −7.6±0.1, −7.8±0.2, and −7.3±0.1 respectively (mean±S.D., n=4-5) (FIG. 5B).

While the invention has been described in detail with reference to certain preferred embodiments thereof, it will be understood that modifications and variations are within the spirit and scope of that which is described and claimed.

### Summary of Sequences

Sequence ID No. 1 is a nucleotide sequence encoding a human tuberoinfundibular peptide of 39 residues and the deduced amino acid sequence of the human tuberoinfundibular peptide of 39 residues (TIP39) (prepolypeptide).

Sequence ID No. 2 is the amino acid sequence of the prepolypeptide human tuberoinfundibular peptide of 39 residues prepolypeptide(TIP39) The mature polypeptide is doubleunderlined.

Sequence ID No. 3 is the amino acid sequence of mouse tuberoinfundibular peptide of 39 residues (TIP39).

Sequence ID No. 11 is the amino acid sequence of a mature rat tuberoinfundibular peptide of 39 residues (TIP39).

Sequence ID No. 12 is the amino acid sequence of a mature bovine tuberoinfundibular peptide of 39 residues (TIP39).

Sequence ID No. 13 is the amino acid sequence of a mature mouse tuberoinfundibular peptide of 39 residues.

Sequence ID No. 14 is the amino acid sequence of a mature human tuberoinfundibular peptide of 39 residues (hTIP39).

What is claimed is:

1. An isolated nucleic acid molecule, comprising a sequence of nucleotides that encodes a human tuberoinfundibular peptide of 39 residues, wherein the sequence of nucleotides is selected from the group consisting of:
(a) a sequence of nucleotides that encodes a human tuberoinfundibular peptide of 39 residues and comprises the sequence of nucleotides set forth in SEQ ID NO:1;
(b) a sequence of nucleotides that encodes a human tuberoinfundibular peptide of 39 residues and that hybridizes under conditions of high stringency to the complement of the sequence of nucleotides set forth in SEQ ID NO:1; and, if it is DNA, is fully complementary or, if it is RNA, is identical to mRNA native to a human cell;
(c) a sequence of nucleotides degenerate with the human tuberoinfundibular peptide of 39 residues encoding sequence of (a) or (b).

2. An isolated nucleic acid molecule, comprising a coding region that encodes a splice variant of a human tuberoinfundubilar peptide of 39 residues, wherein the human tuberoinfundubilar peptide of 39 residues is encoded by a sequence of nucleotides as set forth in SEQ ID NO:1.

3. The isolated nucleic acid molecule according to claim 1, wherein the isolated nucleic acid molecule is genomic DNA.

4. The isolated nucleic acid molecule according to claim 1, wherein said isolated nucleic acid molecule is mRNA.

5. The isolated nucleic acid molecule according to claim 1, wherein said isolated nucleic acid molecule is cDNA.

6. An isolated nucleic acid molecule that encodes a human tuberoinfundibular peptide of 39 residues having an amino acid sequence as set forth in SEQ ID NO: 14.

7. Isolated cells comprising the nucleic acid molecule of claim 1, wherein the cells are bacterial cells, mammalian cells or amphibian oocytes, and the nucleic acid molecule is heterologous to the cells.

8. An isolated human tuberoinfundibular peptide of 39 residues encoded by the nucleic acid molecule of claim 1.

9. A cell membrane preparation comprising a human tuberoinfundibular peptide of 39 residues having a sequence of amino acids as forth in SEQ ID NO: 14 produced by a cell that expresses a recombinant expression vector encoding said a human tuberoinfundibular peptide of 39 residues .

10. A recombinant expression sequence comprising a nucleic acid having a nucleotide sequence encoding a human tuberoinfundibular peptide of 39 residues, wherein the nucleotide sequence comprises the sequence of nucleotides as set forth in SEQ ID NO:1 and expresses said peptide in a transformed culture of eukaryotic or prokaryotic cells.

11. A cell culture transformed with the recombinant expression construct of claim 1.

12. An expression vector comprising the nucleic acid molecule of claim 2, operably linked to a regulatory nucleotide sequence that controls expression of the nucleic acid molecule in a host cell.

13. A method for identifying functional human tuberoinfundibular peptide of 39 residues, said method comprising:
(a) introducing the nucleic acid molecule of claim 1 into a suitable host cell that expresses a functional parathyroid hormone-2 receptor (PTH₂) receptor; and
(b) assaying for parathyroid hormone-2 receptor activity in cells of step (a).

14. A method for identifying DNA sequences encoding a human tuberoinfundibular peptide of 39 residues, the method comprising probing a cDNA library or a genomic library with a labeled probe comprising the nucleotide sequence of SEQ ID NO:1, and recovering from the library those sequences having a significant degree of homology relative to the probe.

15. A method for identifying a human tuberoinfundibular peptide of 39 residues, comprising:
(a) introducing the nucleic acid molecule of claim 1 into eukaryotic cells; and
(b) detecting parathyroid hormone-2 receptor activity in the cells of step (a), wherein the activity is mediated by a polypeptide encoded by the introduced nucleic acid molecule.

16. A method for detecting human tuberoinfundibular peptide of 39 residues messenger RNA in a biological sample comprising the steps of:
(a) contacting all or part of the nucleic acid sequence shown in SEQ ID NO:1 with the biological sample under conditions allowing a complex to form between said nucleic acid sequence and said messenger RNA
(b) detecting said complexes; and determining the level of said messenger RNA.

17. A bioassay for identifying a test compound, which modulates the activity of a human tuberoinfundibular peptide of 39 residues, said bioassay comprising:
(a) measuring the second messenger activity of eukaryotic cells transformed with DNA encoding the human tuberoinfundibular peptide of 39 residues in the absence of the test compound, thereby obtaining a first measurement;
(b) measuring the second messenger activity of eukaryotic cells transformed with DNA encoding the human tuberoinfundibular peptide of 39 residues in the presence of the test compound, thereby obtaining a second measurement; and
(c) comparing the first and second measurement and identifying those compounds that result in a difference between the first measurement and the second measurement as a test compound that modulates the activity of the human tuberoinfundibular peptide of 39 residues, wherein the eukaryotic cells express a functional human parathyroid hormone-2 receptor.

18. A method for following progress of a therapeutic regime designed to alleviate a condition characterized by abnormal expression of a gene product of the isolated nucleic acid molecule of claim 1, comprising:
(a) assaying a sample from a subject to determine level of a parameter selected from the group consisting of (i) a polypeptide encoded by a the nucleotide sequence of SEQ ID NO:1 and (ii) a polypeptide having the amino acid sequence as set forth in SEQ ID NO: 14, at a first time point;
(b) assaying level of the parameter selected in (a) at a second time point and
(c) comparing said level at the second time point to the level determined in (a) as a determination of effect of the therapeutic regime.

19. A method for determining regression, progression or onset of a pathological disorder characterized by a dysfunctional signal transduction comprising incubating a sample obtained from a patient with said disorder with a complimentary nucleic acid hybridization probe having a sequence of nucleotides that are substantially homologous to those of SEQ ID NO:2 and determining binding between said probe and any complimentary mRNA that may be present in said sample as determinative of the regression, progression or onset of said pathological disorder in said patient.

20. The method of claim 19, wherein the sample is a tissue.

21. A method for determining regression, progression or onset of a pathological disorder characterized by a dysfunctional signal transduction comprising: contacting a sample, from a patient with said disorder, with a detectable probe that is specific for the gene product of the isolated nucleic acid molecule of claim 1, under conditions favoring formation of a probe/gene product complex, the presence of which is indicative of the regression, progression or onset of said pathological disorder in said patient.

22. The method of claim 21, wherein the probe is an antibody.

23. The method of claim 22, wherein said antibody is labeled with a radioactive label or an enzyme.

24. A pharmaceutical composition comprising the polypeptide according to claim 6 in combination with a pharmaceutically acceptable carrier, diluent or excipient.

25. A method for preventing or delaying onset of a condition associated with reduced or non-existent levels of the polypeptide of claim 6 in a subject prone thereto comprising administering an effective amount of the polypeptide to the subject sufficient to prevent or delay onset of the condition.

26. A method for monitoring the efficacy of an agent in correcting an abnormal level of the polypeptide of claim 6 in a subject prone thereto, comprising administering an effective amount of said agent to said subject and determining a level of the polypeptide in said subject following its administration, wherein a change in the level of the polypeptide towards a normal level is indicative of the efficacy of said agent.

27. A method for detecting a binding partner for the polypeptide of claim 6 in a sample suspected of containing the binding partner, comprising:
(i) contacting the sample with said polypeptide under conditions favoring binding of said polypeptide to said binding partner;
(ii) determining presence of said binding partner in said sample by detecting binding of said polypeptide to said binding partner.

28. A method of modulating the endogenous signal transducing activity of a parathyroid hormone-2 receptor in a mammal comprising administering to the mammal an effective amount of the polypeptide of claim 6 to modulate the activity.

29. A method for screening and identifying agonists of human tuberoinfundibular peptide of 39 residues, comprising:
(a) contacting a cell line that expresses the human parathyroid hormone-2 receptor with a test compound in the presence and in the absence of a human tuberoinfundibular peptide of 39 residues;
(b) determining whether, in the presence of human tuberoinfundibular peptide of 39 residues, the test compound inhibits the binding of the human tuberoinfundibular peptide of 39 residues to a cell surface receptor in the cell line, and
(c) determining whether, in the absence of the human tuberoinfundibular peptide of 39 residues, the test compound mimics the cellular effects of the tuberoinfundibular peptide of 39 residues on the cell line, in which agonists are identified as those test compounds that inhibit the binding but mimic the cellular effects of human tuberoinfundibular peptide of 39 residues on the cell line.

30. A method for screening and identifying antagonists of human tuberoinfundibular peptide of 39 residues, comprising:
(a) contacting a cell line that expresses human parathyroid hormone-2 receptor with a test compound in the presence of human tuberoinfundibular peptide of 39 residues; and
(b) determining whether the test compound inhibits the binding and cellular effects of human tuberoinfundibular peptide of 39 residues on the cell line, in which antagonists are identified as those compounds that inhibit both the binding and cellular effects of human tuberoinfundibular peptide of 39 residues on the cell line.

31. Method for screening for a disorder characterized by expression of a dysfunctional human tuberoinfundibular peptide of 39 residues coded for by a cDNA comprising a sequence of nucleotides substantially homologous to those set forth in SEQ ID NO:1, comprising contacting a sample from a subject believed to suffer from said disorder with an antibody specific for an expression product of SEQ ID NO:1, and determining binding between said antibody and said expression production as an indication of possible presence of said disorder in said subject.

32. Method for screening for a disorder characterized by expression of a dysfunctional human tuberoinfundibular peptide of 39 residues encoded by a cDNA molecule comprising a sequence of nucleotides substantially homologous to those set forth in SEQ ID NO:1, comprising contacting a cDNA or mRNA containing sample from a subject with a nucleic acid hybridization probe which hybridizes to a cDNA molecule comprising a sequence of nucleotides as set forth in SEQ ID NO:1, and determining binding of said hybridization probe to said cDNA or mRNA as an indication of possible presence of said disorder in said subject.

33. Method for inhibiting binding of human tuberoinfundibular peptide of 39 residues to a cell presenting a parathyroid hormone-2 receptor comprising adding an amount of the antagonist of claim 30 to a sample containing said cell in an amount sufficient to inhibit binding of the human tuberoinfundibular peptide of 39 residues to said cell.

34. An antibody that is specific for the polypeptide of claim 6.

35. The antibody according to claim 34, wherein said antibody is a monoclonal antibody.
